(19) United States
(12) Patent Application Publication
(10) Pub. No.: US 2021/0128791 A1
(43) Pub. Date: May 6, 2021

(54) GALVANIC REDOX MATERIAL AND IMPLANTABLE DEVICE AND METHODS THEREOF

(71) Applicant: THE REGENTS OF THE UNIVERSITY OF CALIFORNIA, Oakland, CA (US)

(72) Inventors: B. Chia Soo, Los Angeles, CA (US); Kang Ting, Los Angeles, CA (US); Zhong Zheng, Van Nuys, CA (US); Benjamin Wu, San Marino, CA (US); Yulong Zhang, Los Angeles, CA (US)

(21) Appl. No.: 17/073,945

(22) Filed: Oct. 19, 2020

Related U.S. Application Data
(63) Continuation of application No. PCT/US2019/027933, filed on Apr. 17, 2019.
(60) Provisional application No. 62/659,093, filed on Apr. 17, 2018.

(51) Int. Cl. A61L 27/54; A61L 27/04; A61L 27/06; A61L 31/16; A61L 27/08; A61L 31/02; A61N 1/20

(52) U.S. Cl. CPC A61L 27/54; A61L 27/042; A61L 27/047; A61L 27/06; A61L 2430/12; A61L 27/08; A61L 31/024; A61N 1/205; A61L 31/16

(57) ABSTRACT

The application discloses an implantable device, comprising a galvanic redox system formed on a body substrate of the implantable device. The implantable device has a non-zero surface potential when it is deployed. The galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed. Methods of making and using the implantabe device are also disclosed.

### CROSS-REFERENCE TO RELATED APPLICATIONS

This application is a continuation of International application No. PCT/US19/27933, filed Apr. 17, 2019, which claims the benefit of U.S. Provisional Patent Application No. US 62/659,093, filed on Apr. 17, 2018. The teaching of these applications is incorporated herein by reference in its entirety.

### FIELD OF THE INVENTION

The invention disclosed herein generally relates to implantable devices.

### BACKGROUND OF THE INVENTION

Tissue integration is a major challenge in the field of implantable biomedical device. Efforts are made to achieve improved soft tissue integration and osteointegration in the biomedical fields that involve implantable devices, e.g., dental implants, stenting, or bone implants with success of some degree, but tissue integration remains a major challenge.

Additionally, implant-associated microbial infections are one of the most serious complications in orthopedic surgery because they are extremely difficult to treat and result in increased morbidity and substantially worse outcomes. Despite a recent focus on aseptic surgical and procedural techniques, catheter- and surgical implant-associated infections account for nearly half of the 2 million cases of nosocomial infections in the United States per year, representing a significant healthcare and economic burden. Devices and methods for imaging sub-millimeter-sized tumors that are embedded in tissues (e.g., at depths greater than 1-2 mm) are not available. Consequently, methods for treating such tumors are also lacking due to the inability in combining high specific and sensitive imaging with highly conformal radiation.’

Management of an implant-associated infection typically requires device removal, multiple debridement surgeries, and long-term systemic antibiotic therapy, despite the associated side effects and additional complications. However, these additional surgical procedures and medical therapies not only increase the healthcare costs, but also result in an increased rate of recurrence, particularly because it is difficult to clear the infection from devascularized bone and other necrotic tissues. Soon after introduction of an implant, a conditioning layer composed of host-derived adhesins (including fibrinogen, fibronectin, collagen, etc.) covers the surface of the implant. This layer promotes adherence of free-floating (planktonic) bacteria, which subsequently form an extracellular anionic polysaccharide 3 dimensional (3D) biofilm. Once a biofilm forms, it is extremely difficult to treat these infections because the biofilm blocks the penetration of both host immune cells (such as macrophages) and systemic antibiotics, promoting further bacterial survival. Given the difficulties in treating implant-associated infections, strategies aimed at preventing the infection and biofilm formation during surgery and in the immediate postoperative period may serve as more effective alternative that can prevent these infections altogether.

Prior studies have coated or covalently-linked antibiotics onto prosthetic materials to prevent bacterial infection during surgical implantation. Although this local antibiotic therapy may be effective, they are limited to certain bacterial species and these infections can be caused by a spectrum of bacteria, including Gram-positive Staphylococcus aureus, Staphylococcus epidermidis and Streptococci species, and Gram-negative Pseudomonas and Enterobacter species. Moreover, antibiotics used in this manner can contribute to the development of antibiotic resistance, which is especially relevant as there is an increasing number of infections caused by methicillin-resistant S. aureus (MRSA) and methicillin-resistant S. epidermidis (MRSE) strains.

What is needed in the art are implant with materials that can resist infection while simultaneously promoting tissue integration and/or regeneration, e.g., bone growth.

The embodiments described below address the above-identified issues and needs.

### SUMMARY OF THE INVENTION

In one aspect of the present invention, it is provided an implantable device, comprising a galvanic redox system formed on a body substrate of the implantable device, the implantable device having a non-zero surface potential when it is deployed,

wherein the galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and the first metal and the second metal form a galvanic redox metal pair (“GRMP”).

In some embodiments of the invention device, optionally in combination with any or all the various embodiments disclosed herein, the non-zero surface potential is a positive surface potential.

In some embodiments of the invention device, optionally in combination with any or all the various embodiments disclosed herein, the first metal is Fe, Al, Mg, Zn, Cu, Cr, Zr, a stainless-steel alloy, a titanium alloy, a cobalt-chromium alloy, amalgam, or combination thereof.

In some embodiments of the invention device, optionally in combination with any or all the various embodiments disclosed herein, the second metal is Ag, Ti, a silver oxide, a titanium oxide, Au, or Pt, or a combination thereof. In some embodiments, the second metal can be replaced in whole in part with Graphite,

In some embodiments of the invention device, optionally in combination with any or all the various embodiments disclosed herein, the implantable device further comprises an antimicrobial component having an optional antimicrobial agent, the antimicrobial component being included in the second metal side of the galvanic redox system or being an additional component deposited on top of the galvanic redox system.

In some embodiments of the invention device, optionally in combination with any or all the various embodiments disclosed herein, the plurality of particles comprising the second metal is inlayed with or embedded within the body substrate of the implantable device or included in a coating formed from a polymer material.

In some embodiments of the invention device, the second metal comprises silver (Ag).

In some embodiments of the invention device, optionally in combination with any or all the various embodiments disclosed herein, the antimicrobial component comprises silver particles.

In some embodiments of the invention device, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention device, optionally in combination with any or all the various embodiments disclosed herein, the plurality of particles comprises silver nanoparticles

In some embodiments of the invention device, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention device, optionally in combination with any or all the various embodiments disclosed herein, the implant device is a dental implant, an orthopedic implant, a stent or a cosmetic implant.

In another aspect of the present invention, it is provided a method of fabricating an implantable device, comprising forming a galvanic redox system formed on a body substrate of the implantable device, the implantable device having a non-zero surface potential when it is deployed,

wherein forming the galvanic redox system comprises forming a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and

the first metal and the second metal form a galvanic redox metal pair (“GRMP”).

In some embodiments of the invention method, optionally in combination with any or all the various embodiments disclosed herein, the non-zero surface potential is a positive surface potential.

In some embodiments of the invention method, optionally in combination with any or all the various embodiments disclosed herein, the first metal is Fe, Al, Mg, Zn, Cu, Cr, Zr, or stainless-steel alloy, titanium alloy, cobalt-chromium alloy, amalgam, or a combination thereof.

In some embodiments of the invention method, optionally in combination with any or all the various embodiments disclosed herein, the second metal is Ag, Ti, a silver oxide, a titanium oxide, Au, Pt, or a combination thereof. In some embodiments, the second metal can be replaced in whole or in part with graphite.

In some embodiments of the invention method, optionally in combination with any or all the various embodiments disclosed herein, the implantable device comprises an antimicrobial component having an optional antimicrobial agent, the antimicrobial component being included in the second metal side of the galvanic redox system or being an additional component deposited on top of the galvanic redox system.

In some embodiments of the invention method, optionally in combination with any or all the various embodiments disclosed herein, the plurality of particles comprising the second metal is inlayed with or embedded within the body substrate of the implantable device or included in a coating formed from a polymer material.

In some embodiments of the invention method, optionally in combination with any or all the various embodiments disclosed herein, the second metal comprises silver (Ag).

In some embodiments of the invention method, optionally in combination with any or all the various embodiments disclosed herein, the antimicrobial component comprises silver particles.

In some embodiments of the invention method, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention method, optionally in combination with any or all the various embodiments disclosed herein, the plurality of particles comprises silver nanoparticles.

In some embodiments of the invention method, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention method, optionally in combination with any or all the various embodiments disclosed herein, the implantable device is a dental implant, an orthopedic implant, a stent or a cosmetic implant.

In another aspect of the present invention, it is provided a method of treating or ameliorating a medical or cosmetic condition in a subject in need thereof, comprising applying an implantable device to the subject, the implantable device comprising a galvanic redox system formed on a body substrate of the implantable device, the implantable device having a non-zero surface potential when it is deployed, wherein the galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and

the first metal and the second metal form a galvanic redox metal pair (“GRMP”).

In some embodiments of the invention method, optionally in combination with any or all the various embodiments disclosed herein, the non-zero surface potential is a positive surface potential.

In some embodiments of the invention method, optionally in combination with any or all the various embodiments disclosed herein, the first metal is Fe, Al, Mg, Zn, Cu, Cr, Zr, a stainless-steel alloy, a titanium alloy, a cobalt-chromium alloy, amalgam, or a combination thereof.

In some embodiments of the invention method, optionally in combination with any or all the various embodiments disclosed herein, the second metal is Ag, Ti, a silver oxide, a titanium oxide, Au, Pt, or a combination thereof. In some embodiments, the second metal can be replaced in whole or in part with graphite.

In some embodiments of the invention method, optionally in combination with any or all the various embodiments disclosed herein, the implantable device comprises an antimicrobial component having an optional antimicrobial agent, the antimicrobial component being included in the second metal side of the galvanic redox system or being an additional component deposited on top of the galvanic redox system.

In some embodiments of the invention method, optionally in combination with any or all the various embodiments disclosed herein, the plurality of particles comprising the second metal is inlayed with or embedded within the body substrate of the implantable device or included in a coating formed from a polymer material.

In some embodiments of the invention method, optionally in combination with any or all the various embodiments disclosed herein, the second metal comprises silver (Ag).

In some embodiments of the invention method, optionally in combination with any or all the various embodiments disclosed herein, the antimicrobial component comprises silver particles.

In some embodiments of the invention method, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention method, optionally in combination with any or all the various embodiments disclosed herein, the plurality of particles comprises silver nanoparticles.

In some embodiments of the invention method, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention method, optionally in combination with any or all the various embodiments disclosed herein, the implantable device is a dental implant, an orthopedic implant, a stent or a cosmetic implant.

In some embodiments of the invention method, optionally in combination with any or all the various embodiments disclosed herein, the subject is a human being.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1A-1C are illustrations of the nanoscale galvanic redox system in the silver nanoparticles (or nanosilver; AgNP or Ag^{NANO})/PLGA-coated matrix on the surface of metal materials.

FIG. 2A-2C show AgNP/PLGA-coated 316L stainless steel alloy (SNPSA) and AgNP/PLGA-coated titanium (SNPT) surface morphologies and surface potentials.

FIG. 3A-3C show Surface morphologies and properties of SNPSA and SNPT after conditional osteogenic medium (COM) treatment.

FIG. 4A-4D show Osteogenic ability of SNPSA and SNPT in vitro with different AgNP proportions (0%,10%, 20%).

FIG. 5A-5D show in vivo osteogenic effects of SNPSA and SNPT in a rat femoral intramedullary rod (FIR) model.

FIG. 6 is an illustration of the Transwell® plate used to perform the COM treatment experiment with SNPSA and SNPT. The MC3T3-E1 cells were cultured on Matrigel pre-coated Transwell® plates with 500 μl of the osteogenic medium in order to avoid direct contact with the surface morphology of the SNPSA and SNPT materials.

FIG. 7A-7B are scan electronic microscopy (SEM) images of the 316L-stainless steel alloy (SA) surface before (A) and after (B) in vivo implantation.

FIG. 8A and FIG. 8B illustrate exemplary SEM images of SNPSA Kirschner (K)-wires. A uniform layer AgNP/PLGA composite was observed on the surface of SA. Aggregates of AgNP were not found in AgNP/PLGA composite layers containing up to 2% AgNP (A). Light microscope images of SNPSA K-wires appear in the top panel. The thickness AgNP/PLGA composite layer was 43.36±0.08 μm (B). Blue box shows the area magnified in the bottom panel. Placing SNPSA K-wires into the pre-reamed intramedullary canal did not considerably damage the coating. White scale bar=100 μm; black scale bar=25 μm.

FIG. 9A-9B and FIG. 9C illustrate exemplary SEM images of AgNP/PLGA coated K-wires before (A) and after bony insertion/removal (B). Note continued adherence of the AgNP/PLGA coating. (C) Light microscope and SEM images of SNPSA discs. A uniform layer of AgNP/PLGA composite without aggregation was observed on the surface of SA. No significant difference was found between 0%- and 2%-SNPSA disc surfaces, while the surfaces of SNPSAs were much smoother than those of uncoated stainless steel alloy discs. Scale bar=50 μm

FIG. 10A-10D exemplary surface free energy of SNPSAs. Dependency of the total surface free energy (a, γₛ), the dispersion component (b, γₛ^{d}), the non-dispersion component (c, γₛ^{nd}) and the polarity (d, γₛ^{nd}/γₛ×100%) on the silver proportion of various SNPSAs during the 9-day incubation in osteogenic medium in vitro. N=6; #, significant difference compared to 0%-SNPSA, ANOVA<0.05; *, significant difference between before and after incubation in osteogenic medium, P<0.05; error bars were too small to show.

FIG. 11A-11D illustrate an exemplary embodiment. In vitro protein adsorption of SNPSAs. Adsorption of the total serum protein (A), bovine serum albumin (BSA) (B), and bone morphogenetic protein (BMP)-2 (C) was measured after 0 and 9 hours of incubation in osteogenic medium. The ratio of protein adsorption of BMP-2/BSA is also shown (d). Data normalized to 0%-SNPSA on day 0. N=6; #, significant difference compared to 0%-SNPSA, ANOVA<0.05; *, significant difference before and after incubation in osteogenic medium, P<0.05; error bars were too small to show.

FIG. 12A-12B illustrate an exemplary embodiment. In vitro antibacterial activity of AgNP/PLGA-coated K-wires (0% Ag^{NANO} and 2% Ag^{NANO}) Different inocula [10³, 10⁴ and 10⁵ colony formation unit (CFU)] of S. aureus Mu50 (A) and Xen36 (B) were incubated in 1 ml broth with AgNP/PLGA-coated K-wires at 37° C. for 1 h to allow adherence of the microorganisms to the AgNP/PLGA-coated K-wire surface. After rinsing with phosphate buffered saline (PBS), AgNP/PLGA-coated K-wires were incubated in 1 ml PBS nutrient for 18 h at 37° C.; 100 μl of the PBS solution was then spread on agar plates for 20 h incubation. The antibacterial effect of AgNP/PLGA-coated K-wires were evaluated with bacterial colony formation after overnight culture.

FIG. 13A-13C illustrate an exemplary embodiment. In vitro bacterial colonization analysis of S. aureus Mu50. Antimicrobial activity of SNPSA against 10³ (A), 10⁴ (B), and 10⁵ (C) CFU S. aureus Mu50 was evaluated. Bacteria were incubated in 1 ml broth with SNPSA K-wires at 37° C. to adherence. At the end of the incubation, bacteria attached to the surface were collected in sterile 0.9% saline solution by sonication for 30 s at 0.6 power with an intermediate size probe and plated onto 10-cm brain-heart infusion broth (BHIB) culture medium plates overnight. After 18 h incubation, the number of colonies on each plate was quantitated following protocols set forth by the U.S. Food and Drug Administration (FDA), for example, in their Bacteriological Analytical Manual and Aerobic Plate Count Method. (accessible at the FDA website, e.g., at www<dot>fda<dot>gov</>Food</>ScienceResearch</>LaboratoryMethods</>BacteriologicalAnalytical ManualBAM</>ucm063346<dot>htm). SNPSA inhibited S. aureus Mu50 initial adherence and extended proliferation in a silver-proportion-dependent manner in vitro. N=4; *, significant difference compared to 0%-SNPSA, ANOVA<0.05; error bars were too small to show.

FIG. 14A-14C illustrate an exemplary embodiment. In vitro bacterial colonization analysis of P. aeruginosa PAO-1. Antimicrobial activity of SNPSAs against 10³ (A), 10⁴ (B), and 10⁵ (C) CFU P. aeruginosa PAO-1 was evaluated. Bacteria were incubated in 1 ml broth with SNPSA K-wires at 30° C. to adherence. At the end of the incubation, bacteria attached to the surface were collected in sterile 0.9% saline solution by sonication for 30 s at 0.6 power with an intermediate size probe and plated onto 10-cm LB culture medium plates overnight. After 18 h incubation, the number of colonies on each plate was quantitated following protocols set forth by the U.S. Food and Drug Administration (FDA), for example, in their Bacteriological Analytical Manual and Aerobic Plate Count Method (accessible at the FDA website, e.g., www<dot>fda<dot>gov</>Food</>ScienceResearch</>LaboratoryMethods</>BacteriologicalAnalytical ManualBAM</>ucm063346<dot>htm). SNPSA inhibited P. aeruginosa PAO-1 initial adherence and extended proliferation in a silver-proportion-dependent manner in vitro. N=4; *, significant difference compared to 0%-SNPSA, ANOVA<0.05; error bars were too small to show.

FIG. 15A-15D illustrate an exemplary embodiment. Ex vivo antibacterial activity of AgNP/PLGA-coated K-wires (0% Ag^{NANO} and 2% Ag^{NANO}). Different inocula (A, C, 10³ CFU and B, D, 10⁵ CFU respectively) of S. aureus Mu50 (A, B) and Xen36 (C, D) were tested with ex vivo model for 18 h incubation at 37° C. After rinsing with PBS, AgNP/PLGA-coated K-wires were incubated in 1 ml PBS nutrient for another 18 h at 37° C.; 100 μl of the PBS solution was then amplified by adding 100 ∞l fresh broth for a 40 h-kinetics test with microplate proliferation assay.

FIG. 16A-16F illustrate an exemplary embodiment. Ex vivo antimicrobial activity of SNPSAs. Using an ex vivo antimicrobial model, antimicrobial activity of SNPSAs against 10³ (A), 10⁴ (B), and 10⁵ (C) CFU S. aureus Mu50, as well as 10³ (D), 10⁴ (E), and 10⁵ (F) CFU P. aeruginosa PAO-1, was evaluated ex vivo. SNPSA effectively inhibited bacterial proliferation in a silver-proportion-dependent manner. N=3; *, significant difference compared to 0%-SNPSA, ANOVA<0.05.

FIG. 17A-17F illustrate an exemplary embodiment. Creation of ex vivo model for AgNP/PLGA-coated K-wires. (A) Isolated mouse femur. (B) Coated K-wires (upper:0% AgNP, lower: 2% AgNP, length: 1 cm). (C) An intramedullary canal was manually reamed into the distal femur with a 25 gauge needle (arrow). (D) An orthopaeadic-grade stainless steel AgNP/PLGA-coated Kirschner wire was then placed in the intramedullary canal (arrow). (E) An inoculum of S. aureus Mu50 or Xen36 in a 2 μl volume was then pipetted into the intramedullary canal and was attached on the nanosilver coated K-wires (arrow). (F) Isolated mouse femur with AgNP/PLGA-coated K-wire and pipetted with 10³ or 10⁵ CFU S. aureus Mu50 or Xen36. Scale bar: 5 mm.

FIG. 18A-18B illustrate an exemplary embodiment. Ex vivo culture model for AgNP/PLGA-coated K-wires. (A) Top view of isolated mouse femur with AgNP/PLGA-coated K-wire injected with 2 μl containing 10³ or 10⁵ CFU S. aureus Mu50 or Xen36 and incubated in 100 μm cell strainers within 6-well cell culture plates. (B) Lateral view of incubation model. The distal femur with the protruding K-wire is angled superiorly so that the proximal femur is in contact with culture medium, while the AgNP/PLGA-coated K-wire does not directly contact the culture medium.

FIG. 19A-19F illustrate an exemplary embodiment. Ex vivo antimicrobial model. Femurs isolated from 12-week old male 129/sv mice (A) were used for SNPSA ex vivo antimicrobial activity test. After locating the femoral intercondylar notch, an intramedullary canal was manually reamed into the distal femur with a 25-gauge needle (B). A SNPSA K-wire was then placed into the intramedullary canal (C) with 2 μl bacteria suspended in PBS (D). These femurs with implants (E) were placed on a 100 μm cell strainer within 6-well culture plate containing 2 ml medium (F). In order to avoid direct contact between SNPSA and cell culture medium, the distal femur with a protruding SNPSA was angled superiorly, and the proximal femur was soaked in culture medium.

FIG. 20A-20B illustrate an exemplary embodiment, demonstrating selective inhibition of fibroblast proliferation over osteoblast proliferation. (A) 5,000 pre-osteoblastic MC3T3-E1 (subclone 4, ATCC CRL-2593) cells were seeded on AgNP/PLGA-composite (NS/PLGA) grafts (red line). After cultured in a-minimal essential medium (a-MEM) supplied with 10% fetal bovine serum (FBS), 1% HT supplement, and 1% penicillin/streptomycin for 4 days at 37° C. with 5% CO, cell viability was evaluated by Vybrand® MTT Cell Proliferation Assay Kit. Up to 2.0% AgNP affected the viability of MC3T3-E1 cells proliferation. (B) 5,000 rat dermal fibroblast Rat2 (ATCC CRL-1764) cells were seeded on AgNP/PLGA composite (NS/PLGA) grafts (red line). After cultured in Dulbecco's Modified Eagle Medium (DMEM) supplied with 10% FBS, and 1% penicillin/streptomycin for 4 days at 37° C. with 5% CO2, cell viability was evaluated by Vybrand® MTT Cell Proliferation Assay Kit. AgNP showed obvious cytotoxicity to fibroblasts. Data were descripted as mean±standard error of mean. N=6; *, P<0.001.

FIG. 21A-21C illustrate an exemplary embodiment. In vitro osteoinductive activity of SNPSAs. 2×10³ pre-osteoblastic MC3T3-E1 murine cells (passage 18, subclone 4, ATCC CRL-2593) were seeded on SNPSA discs with 500 ml osteogenic medium (a-MEM supplied with 10% FBS, 1% HT supplement, 1% penicillin/streptomycin, 50 μg/ml ascorbic acid and 10 mM β-glycerophosphate) in 24-well plates at 37° C., 5% CO2, and 95% humidity. All media for cell culture were purchased from Invitrogen. Cell proliferation was estimated using the Vybrand® MTT Cell Proliferation Assay Kit (Invitrogen). Alkaline phosphatase (ALP) activity and degree of mineralization (assessed by Alizarin Red staining) were used to quantify the effect of SNAPS on osteoblastic differentiation. SNPSAs significantly promoted MC3T3-E1 cell proliferation (A), ALP activity (B), and mineralization (C). Data normalized to 0%-SNPSA on day 9 (B) and on day 15 (C). N=6; *, P<0.05.

FIG. 22A-22B illustrate exemplary radiographic images of uncontaminated 0%- and 2%-SNPSA implants in rat femoral canals (FCs). All surgical procedures were approved by the UCLA Office of Animal Research Oversight (protocol #2008-073). Using aseptic technique, a 25-30 mm longitudinal incision was made over the anterolateral aspect of the left femur of 12-week old male Sprague-Dawley (SD) rats. The femoral shaft was then exposed by separating the vastus lateralis and biceps femoral muscles. Using a micro-driver (Stryker, Kalamazoo, Mich.), four canals were drilled on each femur with 2 mm interface. SNPSA K-wires were implanted into each predrilled canal. The overlying muscle and fascia were closed with 4-0 Vicryl absorbable suture to secure the implant in place. Following surgery, the animals were housed in separate cages and allowed to eat and drink ad libitum. Weight bearing was started immediately postoperatively, and the animals were monitored daily. Buprenorphine was administered for 2 days as an analgesic, but no antibiotic was administered. Three rats were used in every treatment group. No obvious signs of bone formation were shown in rat FCs implanted with 0%-SNPSA up to 8 weeks post-surgery (A). In contrast, radiography revealed significant bone formation (blue arrows) around 2%-SNPSAs implanted in rat FCs (B).

FIG. 23A-23B exemplary radiographic images of contaminated 0%- and 2%-SNPSA implants in rat FCs, based on experiments with 10³ CFU S. aureus Mu50 (A) or P. aeruginosa PAO-1 (B). All surgical procedures were approved by the UCLA Office of Animal Research Oversight (protocol #2008-073). Using aseptic technique, a 25-30 mm longitudinal incision was made over the anterolateral aspect of the left femur of 12-week old SD rats. The femoral shaft was then exposed by separating the vastus lateralis and biceps femoral muscles. Using a micro-driver (Stryker, Kalamazoo, Mich), four canals were drilled on each femur with 2 mm interface. SNPSA K-wires were implanted into each predrilled canal. For bacterial inoculation, 10³ CFU S. aureus Mu50 (A) or P. aeruginosa PAO-1 (B) in 10 μl PBS (10⁵ CFU/ml) was pipetted into the canal before implantation. After inoculation, the overlying muscle and fascia were closed with 4-0 Vicryl absorbable suture to secure the implant in place. Following surgery, the animals were housed in separate cages and allowed to eat and drink ad libitum. Weight bearing was started immediately postoperatively, and the animals were monitored daily. Buprenorphine was administered for 2 days as an analgesic, but no antibiotic was administered. Three rats were used in every treatment group. 10³ CFU S. aureus Mu50 (A) or P. aeruginosa PAO-1 (B) in 10 μl PBS (10⁵ CFU/ml) was pipetted into the canal before implantation for bacterial invasion. Radiographic evidence of osseous destruction (red arrows), without any obvious signs of bone formation up to 8 weeks post-surgery, was detected in the contaminated 0%-SNPSA group. In contrast, significant bone formation surrounding 2%-SNPSAs implanted in rat FCs at week 8 post-implantation (shown as blue arrows in 2D resolution microCT images), without significant osteolysis, was detected. Newly formed bone around 2%-SNPSA implants was highlighted in 3D microCT reconstruction images (blue shading)

FIG. 24A-24E illustrate exemplary histological and immunohistochemical (IHC) analysis of contaminated 0%- and 2%-SNPSA implants in rat FCs at 8 weeks after implantation. 10³ CFU S. aureus Mu50 or P. aeruginosa PAO-1 in 10 μl PBS (10⁵ CFU/ml) was pipetted into the canal before implantation for bacterial invasion. Taylor-modified Brown and Brenn Gram staining (A) and Giemsa staining (B) revealed bacterial persistence (yellow dotted circles) with massive inflammatory cell infiltration (red arrowheads) in the intramedullary tissue around 0%-SNPSA implants in rat FCs. In contrast, no bacterial survival was evident around 2%-SNPSA implants in the same situation, and inflammatory cell infiltration in the intramedullary tissues around the implants was minimal. Consistent with the radiographic analysis, only minimal bone formation around the 0%-SNPSA groups was observed, whereas significant bone formation (blue arrows) was detected around 2%-SNPSA implants, as shown by H&E staining (C), Masson's Trichrome staining (D), and immunostaining of high-intensity OCN signals (F). Yellow scale bar=50 μm (shown in FIG. 24A); red scale bar=100 μm (shown in FIG. 24B); white scale bar=500 μm (shown in FIG. 24C); black scale bar=200 μm (shown in FIG. 24D and FIG. 24E).

### DETAILED DESCRIPTION OF THE INVENTION

### Definitions

Unless otherwise noted, terms are to be understood according to conventional usage by those of ordinary skill in the relevant art.

As used herein, the term “device” encompasses any device that can be placed within a mammal (e.g., a human, a cow, a dog, etc.) via a surgical or otherwise invasive procedure. In some embodiments, the term device is used interchangeably with the term “scaffold”, “fixture” or “implant.”

As used herein, the term “nanoparticle” encompasses small particles having sizes that are often smaller than micrometers. Exemplary nanoparticle configurations include but are not limited to nanoclusters (e.g., having at least one dimension between 1 and 100 nanometers and a narrow size distribution); nanopowders (e.g., agglomerates of ultrafine particles, nanoparticles, or nanoclusters); nanocrystals (nanometer-sized single crystals, or single-domain ultrafine particles, or groups of crystals). In some embodiments, the size of a nanoparticle will be determined by its smallest dimension. It will be understood that the term nanoparticle does not imply that a spherical configuration. For example, silver nanoparticles do not necessarily suggest a spherical or ball-like shape. Indeed, silver nanoparticles can be spherical, fiber-like, branch-like, cluster-like, or of an irregular shape. In this application, the term “nanosilver” is used interchangeably as “silver nanoparticles.”

As used herein, the term “biocompatible” refers to a property of a material characterized by it, or its physiological degradation products, being not, or at least minimally, toxic to living tissue; not, or at least minimally and reparably, otherwise injurious living tissue; and/or not, or at least minimally and controllably, causative of an immunological reaction in living tissue. With regard to salts, both the cation and anion must be biocompatible.

As used herein, the term “biodegradation” includes all means by which a polymer can be disposed of in a patient's body, which includes bioabsorption, resorption, etc. Degradation occurs through hydrolysis, chemical reactions, or enzymatic reactions. Biodegradation can take place over an extended period of time, for example over 2-3 years. The term “biostable” means that the polymer does not biodegrade or bioabsorb under physiological conditions, or biodegrade or bioabsorb very slowly over a very long period of time, for example, over 5 years or over 10 years.

As used herein, the term “layout of the first metal” refers to a configuration of the first metal formed on the body substrate of the implantable device disclosed here, examples of such can be high density discontinuous dots or discrete deposits of the first metal or a thin layer. In this context, high density shall mean 100 or more dots or discrete deposits per 1 cm² (for example, 100, 500, 1,000, 5,000, 10,000, 50,000, 100,000, 500,000, or 1,000,000 dots or discrete deposits per 1 cm²) and a thin layer can be a uniform thin layer or a layer formed by joined or substantially joined dots or deposits of the first metal.

As used herein, the term “coating” is broadly defined as a layer of substance or material that is deposited over a surface of a device (e.g., a scaffold or an implant). In some embodiments, a polymeric matrix comprising silver nanoparticles is deposited as a coating upon a metal or polymeric device. In some embodiments, the coating comprises one or more layers in any combination, with one or more of such layers comprising silver nanoparticles. In some embodiments, multiple layers including but not limited to a primer layer, which may improve adhesion of subsequent layers on the implantable substrate or on a previously formed layer; (b) a reservoir layer, which may comprise a polymer and nanoparticles in the presence or absence a therapeutic agent or, alternatively, a polymer free agent; (c) a topcoat layer, which may serve as a way of controlling the accessibility of the silver nanoparticles or the rate of release of the therapeutic agent; and (d) a biocompatible finishing layer, which may improve the biocompatibility of the coating. In some embodiments, the polymer matrix and polymer substrate can be completely absorbed by the body, preferably at different rate.

As used herein, the term “polymer material” and “polymeric material” can be used interchangeably.

As used herein, the term “is included” shall mean “is a part of or the whole of”.

Unless otherwise noted, terms are to be understood according to conventional usage by those of ordinary skill in the relevant art.

### Implantable Device

In one aspect of the present invention, it is provided an implantable device, comprising a galvanic redox system formed on a body substrate of the implantable device, the implantable device having a non-zero surface potential when it is deployed,

wherein the galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and the first metal and the second metal form a galvanic redox metal pair (“GRMP”).

In some embodiments of the invention device, optionally in combination with any or all the various embodiments disclosed herein, the non-zero surface potential is a positive surface potential.

In some embodiments of the invention device, optionally in combination with any or all the various embodiments disclosed herein, the first metal is Fe, Al, Mg, Zn, Cu, Cr, Zr, a stainless-steel alloy, a titanium alloy, a cobalt-chromium alloy, amalgam, or a combination thereof.

In some embodiments of the invention device, optionally in combination with any or all the various embodiments disclosed herein, the second metal is Ag, Ti, a silver oxide, a titanium oxide, Au, Pt, or a combination thereof. In some embodiments, the second metal can be replaced in whole or in part with graphite.

In some embodiments of the invention device, optionally in combination with any or all the various embodiments disclosed herein, the implantable device further comprises an antimicrobial component having an optional antimicrobial agent, the antimicrobial component being included in the second metal side of the galvanic redox system or being an additional component deposited on top of the galvanic redox system.

In some embodiments of the invention device, optionally in combination with any or all the various embodiments disclosed herein, the plurality of particles comprising the second metal is inlayed with or embedded within the body substrate of the implantable device or included in a coating formed from a polymer material.

In some embodiments of the invention device, the second metal comprises silver (Ag).

In some embodiments of the invention device, optionally in combination with any or all the various embodiments disclosed herein, the antimicrobial component comprises silver particles.

In some embodiments of the invention device, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention device, optionally in combination with any or all the various embodiments disclosed herein, the plurality of particles comprises silver nanoparticles

In some embodiments of the invention device, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention device, optionally in combination with any or all the various embodiments disclosed herein, the implant device is a dental implant, an orthopedic implant, a stent or a cosmetic implant.

In some embodiments, the polymeric material is biocompatible. In some embodiments, the polymeric material is bioabsorbable. In some embodiments, the polymeric material is biodegradable.

In some embodiments, one or more additional coatings can be deposited over the silver-containing nanoparticles or a coating comprising the silver-containing nanoparticles. The additional coating can be formed by one or more polymeric material that is biocompatible, bioabsorbable and/or biodegradable.

Nanoparticles (e.g., of silver or with silver embedded therein) of a wired range of sizes can be used to impart antimicrobial property to a medical device (e.g., an implantable device). In some embodiments, the nanoparticles have a mean size of about 1000 nm or smaller, about 900 nm or smaller, about 800 nm or smaller, about 700 nm or smaller, about 600 nm or smaller, about 500 nm or smaller, about 400 nm or smaller, about 300 nm or smaller, about 250 nm or smaller, about 200 nm or smaller, about 180 nm or smaller, about 150 nm or smaller, about 120 nm or smaller, about 100 nm or smaller, about 90 nm or smaller, about 80 nm or smaller, about 70 nm or smaller, about 60 nm or smaller, about 50 nm or smaller, about 45 nm or smaller, about 40 nm or smaller, about 35 nm or smaller, about 32 nm or smaller, about 30 nm or smaller, about 28 nm or smaller, about 25 nm or smaller, about 22 nm or smaller, about 20 nm or smaller, about 18 nm or smaller, about 15 nm or smaller, about 12 nm or smaller, about 10 nm or smaller, about 8 nm or smaller, about 5 nm or smaller, or about 2 nm or smaller. In some embodiments, the nanoparticles used have a size between 20 nm to 40 nm.

In some embodiments, about 10% or more of the nanoparticles have sizes that are comparable to the mean size of the population of nanoparticles. In some embodiments, about 20% or more of the nanoparticles have sizes that are comparable to the mean size of the population of nanoparticles. In some embodiments, about 30% or more of the nanoparticles have sizes that are comparable to the mean size of the population of nanoparticles. In some embodiments, about 35% or more of the nanoparticles have sizes that are comparable to the mean size of the population of nanoparticles. In some embodiments, about 40% or more of the nanoparticles have sizes that are comparable to the mean size of the population of nanoparticles. In some embodiments, about 45% or more of the nanoparticles have sizes that are comparable to the mean size of the population of nanoparticles. In some embodiments, about 50% or more of the nanoparticles have sizes that are comparable to the mean size of the population of nanoparticles. In some embodiments, about 55% or more of the nanoparticles have sizes that are comparable to the mean size of the population of nanoparticles. In some embodiments, about 60% or more of the nanoparticles have sizes that are comparable to the mean size of the population of nanoparticles. In some embodiments, about 65% or more of the nanoparticles have sizes that are comparable to the mean size of the population of nanoparticles. In some embodiments, about 70% or more of the nanoparticles have sizes that are comparable to the mean size of the population of nanoparticles. In some embodiments, about 75% or more of the nanoparticles have sizes that are comparable to the mean size of the population of nanoparticles. In some embodiments, about 80% or more of the nanoparticles have sizes that are comparable to the mean size of the population of nanoparticles. In some embodiments, about 85% or more of the nanoparticles have sizes that are comparable to the mean size of the population of nanoparticles. In some embodiments, about 80% or more of the nanoparticles have sizes that are comparable to the mean size of the population of nanoparticles. In some embodiments, about 95% or more of the nanoparticles have sizes that are comparable to the mean size of the population of nanoparticles. In some embodiments, about 98% or more of the nanoparticles have sizes that are comparable to the mean size of the population of nanoparticles. In some embodiments, about 99% or more of the nanoparticles have sizes that are comparable to the mean size of the population of nanoparticles.

In some embodiments, a device (e.g., with an antimicrobial coated on its surface or embedded within) provided herein is an osteoconductive scaffold that promotes osteoblastic cell ingrowth and at the same time prevents fibroblastic cell ingrowth. Advantageously, silver nanoparticles are preferentially toxic to fibroblasts rather than osteoblasts.

Exemplary polymeric material that can be used here include but are not limited to a biocompatible or bioabsorbable polymer that is one or more of poly(DL-lactide), poly(L-lactide), poly(L-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof. In some embodiments, the polymeric material comprises a combination of these polymers.

In some embodiments, the polymeric material comprises poly(D,L-lactide-co-glycolide). In some embodiments, the polymeric material comprises poly(D,L-lactide). In some embodiments, the polymeric material comprises poly(L-lactide).

Additional exemplary polymers include but are not limited to poly(D-lactide) (PDLA), polymandelide (PM), poly(lactide-co-glycolide) (PLGA), polylactide (PLA), poly glycolic acid (PGA), polycaprolactone (PCL), poly(3-hydroxybutyrate) (PHB), poly(L-lactide-co-D,L-lactide) (PLDLA), poly(D,L-lactide) (PDLLA), and poly(L-lactide-co-glycolide) (PLLGA), et al., or a combination thereof. With respect to PLLGA, the stent scaffolding can be made from PLLGA with a mole% of GA between 5-15 mol %. The PLLGA can have a mole% of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used. Many other examples can be provided, such as those found in Polymeric Biomaterials, second edition, edited by Severian Dumitriu; chapter 4.

In some embodiments, polymers that are more flexible or that have a lower modulus that those mentioned above may also be used. Exemplary lower modulus bioabsorbable polymers include, polycaprolactone (PCL), poly(trimethylene carbonate) (PTMC), polydioxanone (PDO), poly(3-hydrobutyrate) (PHB), poly(4-hydroxybutyrate) (P4HB), poly(hydroxyalkanoate) (PHA), and poly(butylene succinate), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of a blend can include 1-5 wt % of low modulus polymer.

More exemplary polymers include but are not limited to at least partially alkylated polyethyleneimine (PEI); at least partially alkylated poly(lysine); at least partially alkylated polyornithine; at least partially alkylated poly(amido amine), at least partially alkylated homo- and co-polymers of vinylamine; at least partially alkylated acrylate containing aminogroups, copolymers of vinylamine containing aminogroups with hydrophobic monomers, copolymers of acrylate containing aminogroups with hydrophobic monomers, and amino containing natural and modified polysaccharides, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins, and mixtures thereof.

Additional examples of biocompatible biodegradable polymers include, without limitation, polycaprolactone, poly(L-lactide), poly(D,L-lactide), poly(D,L-lactide-co-PEG) block copolymers, poly(D,L-lactide-co-trimethylene carbonate), poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, polyalkylene oxalates, polyphosphazenes, PHA-PEG, and combinations thereof. The PHA may include poly(α-hydroxyacids), poly(β-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate), MED610, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

In some embodiments, polyethylene is used to construct at least a portion of the device. For example, polyethylene can be used in an orthopedic implant on a surface that is designed to contact another implant, as such in a joint or hip replacement. Polyethylene is very durable when it comes into contact with other materials. When a metal implant moves on a polyethylene surface, as it does in most joint replacements, the contact is very smooth and the amount of wear is minimal. Patients who are younger or more active may benefit from polyethylene with even more resistance to wear. This can be accomplished through a process called crosslinking, which creates stronger bonds between the elements that make up the polyethylene. The appropriate amount of crosslinking depends on the type of implant. For example, the surface of a hip implant may require a different degree of crosslinking than the surface of a knee implant.

Additional examples of polymeric materials can be found, for example, in U.S. Pat. No. 6,127,448 to Domb, entitled “Biocompatible Polymeric Coating Material;” US Pat. Pub. No. 2004/0148016 by Klein and Brazil, entitled “Biocompatible Medical Device Coatings;” US Pat. Pub. No. 2009/0169714 by Burghard et al., entitled “Biocompatible Coatings for Medical Devices;” U.S. Pat. No. 6,406,792 to Briquet et al., entitled “Biocompatible Coatings;” US Pat. Pub. No. 2008/0003256 by Martens et al., entitled “Biocompatible Coating of Medical Devices;” each of which is hereby incorporated by reference herein in its entirety.

In some embodiments, a portion of or the entire device is formed by one or more the aforementioned polymeric materials provided herein. In some embodiments, the polymeric material used to form the device further comprises an antimicrobial agent such that the antimicrobial agent is embedded as a part of the device itself. In some embodiments, a biomedical material such as titanium, silicone or apatite is used to modify the surface of the device such that the device is biocompatible and does not trigger adverse reactions in a patient (e.g., a recipient of an implant).

In some embodiments, a portion of or the entire device is made from a metal material. Exemplary metal materials include but are not limited to stainless steel, chromium, a cobalt-chromium alloy, Tantalum, titanium, a titanium alloy and combinations thereof.

Stainless steel is a very strong alloy, and is most often used in implants that are intended to help repair fractures, such as bone plates, bone screws, pins, and rods. Stainless steel is made mostly of iron, with other metals such as chromium or molybdenum added to make it more resistant to corrosion. There are many different types of stainless steel. The stainless steels used in orthopedic implants are designed to resist the normal chemicals found in the human body. Cobalt-chromium alloys are also strong, hard, biocompatible, and corrosion resistant. These alloys are used in a variety of joint replacement implants, as well as some fracture repair implants, that require a long service life. While cobalt-chromium alloys contain mostly cobalt and chromium, they also include other metals, such as molybdenum, to increase their strength. Titanium alloys are considered to be biocompatible. They are the most flexible of all orthopedic alloys. They are also lighter weight than most other orthopedic alloys. Consisting mostly of titanium, they also contain varying degrees of other metals, such as aluminum and vanadium. Pure titanium may also be used in some implants where high strength is not required. It is used, for example, to make fiber metal, which is a layer of metal fibers bonded to the surface of an implant to allow the bone to grow into the implant, or cement to flow into the implant, for a better grip. Tantalum is a pure metal with excellent physical and biological characteristics. It is flexible, corrosion resistant, and biocompatible.

It will be understood by one of skill in the art that the method and composition provided herein can be used to impart antimicrobial and/or any other advantageous property to any device that is used as a surgical implant. In some embodiments, devices provided herein include medical implants, scaffolds and/or surgical instruments. Exemplary medical implants include but are not limited to stents, balloons, valves, pins, rods, screws, discs, and plates. Exemplary medical implants include but are not limited to an artificial replacement of a body part such as a hip, a joint, etc.

In some embodiments, the devices include an implantable intervertebral device (e.g., a cervical fusion device).

In some embodiments, devices disclosed herein include those associated with dental surgeries, including but not limited to a disc, a bridge, a retainer clip, a screw, a housing, a bone graft, and/or a crown.

In some embodiments, devices disclosed herein include those associated with orthopedic surgeries, including, for example, intramedullary rods, temporary and permanent pins and implants, bone plates, bone screws and pins, and combinations thereof.

In some embodiments, a device provided herein further comprises a bioactive agent such as a graft, an osteoconductive or osteoinductive graft material, a bone morphogenetic protein, a growth factor and a buffer material. Exemplary osteoconductive or osteoinductive graft materials include but are not limited to hydroxyapatite BMP, growth factors (e.g., transforming growth factor (TGF) beta-1,2 and 3, BMP-2, BMP-3, BMP-7, insulin-like growth factor (IGF)-1, and possibly vascular endothelial growth factor (VEGF), neural EGFL like 1 (NELL-1), hydroxyapatite or calcium phosphate.

Additional information on implantable medical devices and osteoinductive materials can be found, for example, in United States Patent Publication No. 2009/0012620 by Youssef J., et al. and entitled “Implantable Cervical Fusion Device;” U.S. Pat. No. 5,348,026 to Davidson and entitled “Osteoinductive Bone Screw;” Barradas A. et al., 2011, “Osteoinductive Biomaterials: Current Knowledge of Properties, Experimental Models and Biological Mechanisms,” European Cells and Materials 21:407-429; U.S. Pat. No. 7,485,617 to Pohl J. et al. and entitled “Osteoinductive Materials,” United States Patent Publication No. 2011/0022180 by Melkent A., et al. and entitled “Implantable Medical Devices;” United States Patent Publication No. 2005/0010304 by Jamali, A. and entitled “Device and Method for Reconstruction of Osseous Skeletal Defects;” United States Patent Publication No. 2010/0036502 by Svrluga R. et al. and entitled “Medical Device for Bone Implant and Method for Producing Such Device;” U.S. Pat. No. 5,672,177 to E. Seldin and entitled “Implantable Bone Distraction Device;” and U.S. Pat. No. 4,611,597 to W. Kraus and entitled “Implantable Device for the Stimulation of Bone Growth;” each of which is hereby incorporated by reference in its entirety.

In some embodiments, the polymeric material forms a coating on the device before an antimicrobial agent is subsequently deposited.

In some embodiments, the antimicrobial agent is dispersed in the polymeric material before the mixture is deposited on the device to form a coating.

In some embodiments, the antimicrobial agent is dispersed in the polymeric material before the mixture is used to form a portion of the device or the entire device itself

In some embodiments, the antimicrobial agent constitutes about 0.1% or less by weight, about 0.2% or less by weight, about 0.3% or less by weight, about 0.4% or less by weight, about 0.5% or less by weight, about 0.6% or less by weight, about 0.7% or less by weight, about 0.8% or less by weight, about 0.9% or less by weight, about 1.0% or less by weight, about 1.1% or less by weight, about 1.2% or less by weight, about 1.3% or less by weight, about 1.4% or less by weight, about 1.5% or less by weight, about 1.6% or less by weight, about 1.7% or less by weight, about 1.8% or less by weight, about 1.9% or less by weight, about 2.0% or less by weight, about 2.1% or less by weight, about 2.2% or less by weight, about 2.3% or less by weight, about 2.4% or less by weight, about 2.5% or less by weight, about 2.6% or less by weight, about 2.7% or less by weight, about 2.8% or less by weight, about 2.9% or less by weight, about 3.0% or less by weight, about 3.2% or less by weight, about 3.5% or less by weight, about 3.8% or less by weight, about 4.0% or less by weight, about 4.5% or less by weight, about 5.0% or less by weight, about 7.0% or less by weight, about 10.0% or less by weight, about 15.0% or less by weight, about 20.0% or less by weight, about 30.0% or less by weight, about 40.0% or less by weight, about 50.0% or less by weight of the total weight of the mixture.

In some embodiments, the antimicrobial agent constitutes about 0.1% or less by weight, about 0.2% or less by weight, about 0.3% or less by weight, about 0.4% or less by weight, about 0.5% or less by weight, about 0.6% or less by weight, about 0.7% or less by weight, about 0.8% or less by weight, about 0.9% or less by weight, about 1.0% or less by weight, about 1.1% or less by weight, about 1.2% or less by weight, about 1.3% or less by weight, about 1.4% or less by weight, about 1.5% or less by weight, about 1.6% or less by weight, about 1.7% or less by weight, about 1.8% or less by weight, about 1.9% or less by weight, about 2.0% or less by weight, about 2.1% or less by weight, about 2.2% or less by weight, about 2.3% or less by weight, about 2.4% or less by weight, about 2.5% or less by weight, about 2.6% or less by weight, about 2.7% or less by weight, about 2.8% or less by weight, about 2.9% or less by weight, about 3.0% or less by weight, about 3.2% or less by weight, about 3.5% or less by weight, about 3.8% or less by weight, about 4.0% or less by weight, about 4.5% or less by weight, about 5.0% or less by weight, about 7.0% or less by weight, about 10.0% or less by weight, about 15.0% or less by weight, about 20.0% or less by weight, about 30.0% or less by weight, about 40.0% or less by weight, about 50.0% or less by weight of the total weight of the polymeric material.

In some embodiments, the device has more than one contact surfaces. It will be understood that the antimicrobial agent can be deposited on a portion of any one or all of these contact surfaces at any percentage as disclosed herein.

In some embodiments, an antimicrobial agent (e.g., alone or in combination with a polymeric material) is deposited upon a contact surface of the device, continuously or discontinuously. For example, an antimicrobial agent (e.g., alone or in combination with a polymeric material) can be deposited continuously over less than about 2%, less than about 5%, less than about 8%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 98%, less than about 99% of a contact surface of the device.

In some embodiments, an antimicrobial agent (e.g., alone or in combination with a polymeric material) can be deposited discontinuously over a contact surface of the device; for example the antimicrobial agent can be deposited over the contact surface as discrete dots, circles, squares, triangles, ovals, or in any other suitable forms or pattern, rendering a total surface area being covered of less than about 2%, less than about 5%, less than about 8%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 98%, less than about 99% of a contact surface of the device.

In some embodiments, one or more therapeutic agents are embedded or impregnated in a device provided herein. In some embodiments, one or more therapeutic agents are embedded or impregnated the polymeric material that forms the device itself or a coating on the surface of a device. In some embodiments, one or more therapeutic agents are added as an additional coating over silver nanoparticles or a coating comprising the silver nanoparticles. In some embodiments, the therapeutic agent can be mixed or dispersed in part of or throughout the polymer scaffold or implant.

It will be understood that any therapeutic agent can be used in combination with the silver nanoparticles provided herein. Exemplary therapeutic agents include but are not limited to one or more anti-microbial agents: aminoglycosides (such as amikacin, gentamicin, kanamycin, neomycin, netilmicin, tobramycin, and/or paromomycin); ansamycins (such as geldanamycin and/or herbimycin); carbacephem (such as loracarbef), carbapenems (such as ertapenem, doripenem, imipenem/cilastatin, and/or meropenem); cephalosporins (such as cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil and/or ceftobiprole); glycopeptides (such as teicoplanin, vancomycin and/or telavancin); lincosamides (such as clindamycin and/or lincomycin); lipopeptide such as daptomycin; macrolides (such as azithromycin, clarithromycin, dirithromycin, erythromycin, roxithromycin, troleandomycin, telithromycin, spectinomycin, spiramycin); monobactams (such as aztreonam, nitrofurans, furazolidone and/or nitrofurantoin), penicillins or penicillin combinations (such as amoxicillin, ampicillin, azlocillin, carbenicillin, cloxacillin, dicloxacillin, flucloxacillin, mezlocillin, methicillin, nafcillin, oxacillin, penicillin v, piperacillin, penicillin g, temocillin, ticarcillin, amoxicillin/clavulanate, ampicillin/sulbactam, piperacillin/tazobactam and/or ticarcillin/clavulanate); polypeptides (such as bacitracin, colistin, and/or polymyxin b); quinolones (such as ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin and/or temafloxacin); sulfonamides (such as mafenide, sulfonamidochrysoidine, sulfacetamide, sulfadiazine, silver sulfadiazine, sulfamethizole, sulfamethoxazole, sulfanilimide, sulfasalazine, sulfisoxazole and/or trimethoprim-sulfamethoxazole-co-trimoxazole); tetracyclines (such as demeclocycline, doxycycline, minocycline, oxytetracycline and/or tetracycline); drugs against mycobacteria such as clofazimine, dapsone, capreomycin, cycloserine, ethambutol, ethionamide, isoniazid, pyrazinamide, rifampicin, rifabutin, rifapentine, streptomycin); arsphenamine, chloramphenicol, fosfomycin, fusidic acid, linezolid, metronidazole, mupirocin, platensimycin, quinupristin/dalfopristin, rifaximin, thiamphenicol, tigecycline, tinidazole, trimethoprim, or combinations thereof.

Exemplary therapeutic agents also include but are not limited to one or more anti-inflammatory agents or any other agents that can be beneficial for the healing of the surgical site or promoting desired growth and development.

In some embodiments, one or more bioactive agents are embedded or impregnated in a device provided herein. In some embodiments, one or more bioactive agents are embedded or impregnated the polymeric material that forms the device itself or a coating on the surface of a device.

In some embodiments, one or more bioactive agents are associated with a device provided herein. In some embodiments, one or more bioactive agents are contained in a compartment of the device.

Exemplary bioactive agents include but are not limited to cells, a biocompatible buffer, growth media or extracellular matrices, growth factors, cytokines, includes metabolites, any small molecules or macromolecules.

In some embodiments, embryonic stem cells (e.g., blastocyst-derived) are cultured and produced within an implantable device as disclosed herein. In some embodiments, blastocyst-derived stem cells isolated from the inner cell mass of blastocysts can be used. In some embodiments, adult stem cells or somatic stem cells, which are found in various tissues (e.g., from bone marrow derived sources), can also be used. Additional adult stem cells include but are not limited to hematopoietic stem cells, mammary stem cells, intestinal stem cells, mesenchymal stem cells, endothelial stem cells, neural stem cells, olfactory adult stem cells, neural crest stem cells, and testicular cells.

In some embodiments, non-stem cells are used. Potentially, all of the 200 or so mammalian cell types within the body can be used in an implantable device as disclosed herein. Exemplary cells include but are not limited to, for example, cells found within a non-embryonic adult, such as insulin secreting cells (e.g., from adults or cadavers) or hepatocytes; islets of Langerhands; cells via somatic cell nuclear transfer (SCNT cells); cells via induced pluripotent stem cells (iPSs cells) either derived by genetic or chemical means; and cells from umbilical cord blood (UCB) cells.

In some embodiments, donor cells are used, including autologous (self) cells or non-autologous cells (e.g., allogenic or xenogenic cells from unrelated donors or other species).

In some embodiments, the cells or tissue used in the device can be suspended in a liquid trapped within a sub-compartment, adhered to the inner walls of the compartment or immobilized on an appropriate support structure provided within the compartment. For example, the cells can be embedded in a gel matrix (e.g., agar, alginate, chitosan, polyglycolic acid, polylactic acid, and the like). In some embodiments, a porous scaffold (e.g., an alignate scaffold) can be used to seed the content within a compartment or sub-compartments of an implantable device. In some embodiments, microcapsules or microbeads can be used to encapsulate or capture cells in the cellular compartment.

In some embodiments, a commercially available growth medium or matrix for mammalian cells is used. For example, Matrigel™ is the trade name for a gelatinous protein mixture secreted by Engelbreth-Holm-Swarm (EHS) mouse sarcoma cells and marketed by BD Biosciences and by Trevigen Inc. under the name Cultrex BME. This mixture resembles the complex extracellular environment found in many tissues and is used by cell biologists as a substrate for cell culture. Components of a standard growth medium or matrix for mammalian cells include but are not limited to extracellular matrix components, growth factors, various cytokines, and one or more pharmaceutical agents, as listed in Table 1.

TABLE 1
Compositions of exemplary biochemical composition.
Extracellular Matrix components
Undefined media
Extract from the EHS tumor (e.g., Matrigel ™
from BD Biosciences)
Growth Factor Reduced Matrigel ™
High Concentration Matrigel ™
Exemplary individual components:
Laminin
Entactin 1
Collagens I-VT
Heparin sulfate proteoglycans
agar
alginate
chitosan
polyglycolic acid
polylactic acid

Exemplary growth factors include but are not limited to adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF) 1, 2, 3, glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta(TGF-β), tumor necrosis factor-alpha (TNF-α), vascular endothelial growth factor (VEGF), placental growth factor (P1GF), fetal bovine somatotrophin (FBS), IL-1, IL-2, IL-3, IL-4, IL-5, IL-6, IL-7, IL-17, and neural EGFL like 1 (NELL-1).

Exemplary small chemical molecules include any chemical compounds, including inorganic and organic compounds, for example, formaldehyde, acetylsalicylic acid, methanol, ibuprofen, and statins. Exemplary macromolecules include but are not limited to monoclonal and polyclonal antibodies, nucleic acid, lipid, fatty acid, and insulin.

In some embodiments, devices provided herein can be placed within any animal, including but not limited to a mammal (e.g., a human, a cow, a dog, a cat, a goat, a sheep, a monkey, a horse, a dolphin, a lion, a tiger, a rat, a mouse, an elephant, and etc.) via a surgical or otherwise invasive procedure.

In some further embodiments, particles of the second metal of the present invention of galvanic redox system can take any shape including spheres, cubic, wire, etc. In some embodiments, a metal composition that have a different electrode potential with the metal substrate, insulator or semiconductor polymers (or their mixtures) complex, can work as the electrodic sites (such as cathodic sites). The other part of the galvanic redox system is a metal substrate with different electrode potential, working as the other electrodic site (such as anodic site). The metal substrate also can be coated first with a specific metal (s first metal) in order to form the specific electrodic sites. The combinations between the cathodic and anodic sites are flexible to choose any kind of metals to form the galvanic redox system, so as to take advantages of the corresponding metal properties to prepare a biomedical device.

Some examples of the combination of the first metal and second metal for the galvanic redox system of invention are stainless/silver, zinc/silver, zirconium/silver, as the electrode potential of zinc is −0.76V, and that of zirconium is −1.45 V, both are significantly lower than that of silver (+0.799 V). Many dental implant alloys can be formed of metals with different electrode potentials, which can be made to have an increased osseointegration by making use of the galvanic redox system of invention. For examples, the Ti/Cr and Ti/Al were used in dental implants, the electrode potential of Al is −1.66V, Cr is −0.73, which is smaller than the potential of titanium (+0.06 V).

### Fabrication of the Devices

In another aspect of the present invention, it is provided a method of fabricating an implantable device, comprising forming a galvanic redox system formed on a body substrate of the implantable device, the implantable device having a non-zero surface potential when it is deployed,

wherein forming the galvanic redox system comprises forming a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and

the first metal and the second metal form a galvanic redox metal pair (“GRMP”).

The method of claim 13, wherein the non-zero surface potential is a positive surface potential.

In some embodiments of the invention method, optionally in combination with any or all the various embodiments disclosed herein, the first metal is Fe, Al, Mg, Zn, Cu, Cr, Zr, a stainless-steel alloy, a titanium alloy, a cobalt-chromium alloy, amalgam, or a combination thereof.

In some embodiments of the invention method, optionally in combination with any or all the various embodiments disclosed herein, the second metal is Ag, Ti, a silver oxide, a titanium oxide, Au, Pt, or a combination thereof. In some embodiments, the second metal can be replaced in whole or in part with graphite.

In some embodiments of the invention method, optionally in combination with any or all the various embodiments disclosed herein, the implantable device comprises an antimicrobial component having an optional antimicrobial agent, the antimicrobial component being included in the second metal side of the galvanic redox system or being an additional component deposited on top of the galvanic redox system.

In some embodiments of the invention method, optionally in combination with any or all the various embodiments disclosed herein, the plurality of particles comprising the second metal is inlayed with or embedded within the body substrate of the implantable device or included in a coating formed from a polymer material.

In some embodiments of the invention method, optionally in combination with any or all the various embodiments disclosed herein, the second metal comprises silver (Ag).

In some embodiments of the invention method, optionally in combination with any or all the various embodiments disclosed herein, the antimicrobial component comprises silver particles.

In some embodiments of the invention method, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention method, optionally in combination with any or all the various embodiments disclosed herein, the plurality of particles comprises silver nanoparticles.

In some embodiments of the invention method, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention method, optionally in combination with any or all the various embodiments disclosed herein, the implantable device is a dental implant, an orthopedic implant, a stent or a cosmetic implant.

In some embodiments of the invention method, optionally in combination with any or all the various embodiments disclosed herein, fabrication of the galvanic redox system can include, but is not limited to, a technique such as electro-spray coating, electrospinning coating, simple dip coating, layer by layer coating, 3D coating, vapor deposition coating, anodizing coating, ion beam coating, plasma spraying, powder coating, extrusion coating, or sandblast coating, etc. These techniques are well known in the art. Detailed description of such techniques is of readily available public domain knowledge and is omitted for concise description of the invention.

### Methods of Use

In another aspect of the present invention, it is provided a method of treating or ameliorating a medical or cosmetic condition in a subject in need thereof, comprising applying an implantable device to the subject, the implantable device comprising a galvanic redox system formed on a body substrate of the implantable device, the implantable device having a non-zero surface potential when it is deployed,

wherein the galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and

wherein:

the first metal site is a layout of the first metal formed on the body substrate or the body substrate itself comprising the first metal;

the second metal site comprises a plurality of particles comprising the second metal; and

the first metal and the second metal form a galvanic redox metal pair (“GRMP”).

The method of claim 13, wherein the non-zero surface potential is a positive surface potential.

In some embodiments of the invention method, optionally in combination with any or all the various embodiments disclosed herein, the first metal is Fe, Al, Mg, Zn, Cu, Cr, Zr, a stainless-steel alloy, a titanium alloy, a cobalt-chromium alloy, amalgam, or a combination thereof.

In some embodiments of the invention method, optionally in combination with any or all the various embodiments disclosed herein, the second metal is Ag, Ti, a silver oxide, a titanium oxide, Au, Pt, or a combination thereof. In some embodiments, the second metal can be replaced in whole or in part with graphite.

In some embodiments of the invention method, optionally in combination with any or all the various embodiments disclosed herein, the implantable device comprises an antimicrobial component having an optional antimicrobial agent, the antimicrobial component being included in the second metal side of the galvanic redox system or being an additional component deposited on top of the galvanic redox system.

In some embodiments of the invention method, optionally in combination with any or all the various embodiments disclosed herein, the plurality of particles comprising the second metal is inlayed with or embedded within the body substrate of the implantable device or included in a coating formed from a polymer material.

In some embodiments of the invention method, optionally in combination with any or all the various embodiments disclosed herein, the second metal comprises silver (Ag).

In some embodiments of the invention method, optionally in combination with any or all the various embodiments disclosed herein, the antimicrobial component comprises silver particles.

In some embodiments of the invention method, optionally in combination with any or all the various embodiments disclosed herein, the GRMP is selected from stainless-steel/silver, zinc/silver, zirconium/silver, chromium/titanium, aluminum/titanium, steel alloy/titanium, stainless steel/gold, stainless steel/graphite.

In some embodiments of the invention method, optionally in combination with any or all the various embodiments disclosed herein, the plurality of particles comprises silver nanoparticles.

In some embodiments of the invention method, optionally in combination with any or all the various embodiments disclosed herein, the polymer material comprises poly(lactide-co-glycolide) (PLGA), polylactide (PLA), poly glycolic acid (PGA), polycaprolactone (PCL), poly(3-hydroxybutyrate) (PHB), et al., or a combination thereof.

In some embodiments of the invention method, optionally in combination with any or all the various embodiments disclosed herein, the implantable device is a dental implant, an orthopedic implant, a stent or a cosmetic implant.

In some embodiments of the invention method, optionally in combination with any or all the various embodiments disclosed herein, the subject is a human being.

In some embodiments, the devices (e.g., a scaffold, a fixture, or an implant) can be used to mask other reagents that may possibly cause microbial infection. In some embodiments, a device can be used to introduce cells or tissues into a mammalian recipient; for example, a carrier of stem cell material. In some embodiments, the devices further include materials that will support or promote the growth and/or development of such cells or tissues.

### Commercial Advantages

Unlike the previous reported electrostimulation generated by external machines, our invention generated built-in electroforce on commercial implant surface to establish the internal electrostimulation of the implanted metal materials themselves, which precisely functions on the interface of the implants and body so as to improve osseointegration of the implantable device of invention.

Meanwhile, since the generation of the nanoscale galvanic redox system is technically high-translational, this technology can be used in deep bone tissue as well as the joints such as the knee, hip, and the shoulders where conventional external electrostimulation techniques are not applicable due to variation of the body shape causing difficulties of electrostimulation.

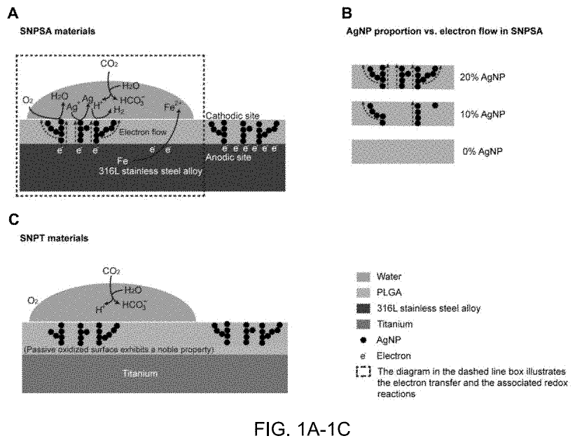

Additionally, the biological interface between an orthopedic implant and the surrounding host tissue has critical effects on clinical outcome. Implant loosening, fibrous encapsulation, corrosion, infection, and inflammation, as well as physical mismatch may have deleterious clinical effects. By employing the electrochemical theory of galvanic reduction-oxidation (redox), we generate a nanoscale galvanic redox system on the surface of metal materials to establish a surface potential that enhances the osseointegration of the metal materials (FIG. 1A-1C).

### A General Process

The following outlines a general process in accordance with the present invention.

### Forming Galvanic Redox System

The fabrication of the galvanic redox system could be but not limited to electro-spray coating, electrospinning coating, simple dip coating, layer by layer coating, 3D coating, vapor deposition coating, anodizing coating, ion beam coating, plasma spraying, powder coating, extrusion coating, sandblast coating, etc. The metal couples to form the galvanic redox system could be any two metals having different electrode potentials.

Beside the stainless/silver, they also could be zinc/silver, zirconium/silver, because the electrode potential of zinc is −0.76V, and zirconium is −1.45 V, both are significantly lower than that of silver (+0.799 V). Many dental implant alloys have metals with different electrode potentials, which could be used to Osseointegration by this galvanic redox theory. For examples, the Ti/Cr and Ti/Al were used in dental implants, the electrode potential of Al is −1.66V, Cr is −0.73, which is smaller than the potential of titanium (+0.06 V).

FIG. 1A-1C are mechanism illustrations of an example of a new biomaterial that employ the galvanic redox theory by the AgNP/PLGA-coated surface of metal materials: a. The positive surface potential of the AgNP/PLGA-coated 316L-SA (SNPSA) is generated by the galvanic process, in which the iron (Fe) in 316L-SA is oxidized to Fe2+, and the released electrons (e−) transfer to the cathodes comprised of silver nanoparticles (AgNPs). Meanwhile, the H+, Ag+, and O2 are reduced on the cathodic sites of SNPSA materials in a moist environment. A positive surface potential and an associated electric field around the cathodic sites are established. b. The electron flow positively correlates with the AgNP proportions in the PLGA layer. In comparison to the 10% AgNP proportion, the 20% AgNP proportion has more AgNP that can connect together to form the electron transduction routes, which can lead to more electron flow and results in both a higher surface potential and osteogenic ability. c. Due to the noble metal property of the passive oxidized titanium surface, the titanium substrate and AgNPs cannot undergo redox reactions on the AgNP/PLGA-coated titanium (SNPT), even when the AgNP/PLGA-coating of the SNPT and SNPSA have the same composition and morphology.

Synthesis of the nanosilver particle-PLGA coating: Nanosilver particles between 20 nm and 40 nm silver particles (QSI-Nano® Silver) were obtained from QuantumSphere, Inc. (Santa Ana, Calif.). The nanosilver-PLGA coating is manufactured using a solvent casting technique known in the art. Briefly, the desired amount of nanosilver will be mixed with 17.5% (w/v) PLGA [85:15 poly(lactic-co-glycolic acid, inherent viscosity: 0.64 dl/g in chloroform; Durect Co., Pelham, Ala.]-chloroform solution. The concentration of silver refers to the weight ratio of nanosilver mixed with PLGA.

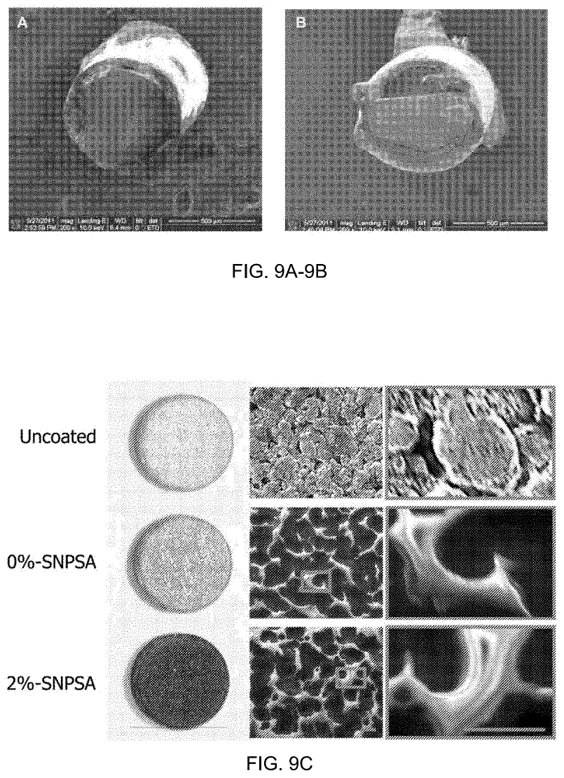

Coating nanosilver PLGA onto titanium implants: The nanosilver/PLGA solution will be layered only onto titanium K-wire implants by immersion with a 5 minute interval between applications of each nanosilver PLGA layer. A 3-layer nanosilver/PLGA coating construct can be initially tested. The coated K-wires will be dried at 37° C. for at least 12 hours before use as we previously described6. We have successfully coated the nanosilver PLGA on K-wires (FIG. 9A-9B, FIG. 9C).

In vitro antimicrobial activity: In vitro antimicrobial activity of nanosilver particle-PLGA coatings will be determined using a standardized microplate proliferation assay as known in the art. Briefly, the nanosilver/PLGA coatings will be incubated with different logarithmic concentration of S. aureus in 200 μl of BHIB in 96-well plates at 37° C. for 1 h to allow adherence of the S. aureus to the coated K-wires. After incubation, coated K-wires will be rinsed with PBS to remove loosely attached bacteria, and then re-cultured in broth for 18 h at 37° C. in another 96-well microplate. During this second incubation step, the viable bacteria attached to the surface of the implants will start to multiply, releasing CFU into the wells. After removal of the implants, 100 μl of released bacteria will be transferred into another 96-well plate and then amplified by adding 100 μl of fresh broth for another 40 h at 37° C. Proliferation of the released cells will be measured at a wavelength of 595 nm using a microplate reader (Tecan, Durham, N.C.) to generate a time-proliferation curve. The coatings with the most potent antimicrobial activity will be evaluated in vivo.

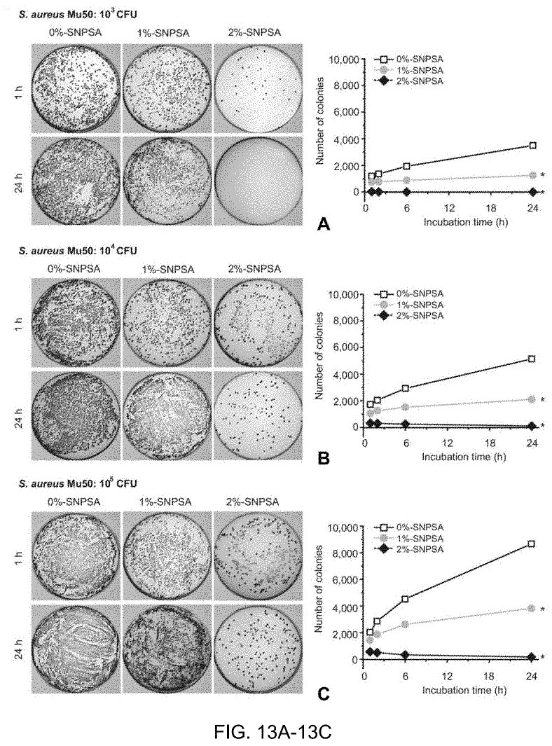

In vivo efficacy of nanosilver/-PLGA coatings. Different characterization techniques can be used to determine the most efficacious nanosilver/PLGA coating. For example, a mouse model of orthopedic implant infection with the endpoints i-iii: (i) In vivo bioluminescence imaging to measure bacterial burden; (ii) Biofilm formation and adherent bacteria; and (iii) Infection-induced inflammation. Nanosilver/PLGA coatings will be evaluated against an intermediate S. aureus inoculum (e.g. 1×10³ CFU) that consistently produces an infection and biofilm formation on the implant and is detectable for 6 post-operative weeks. The nanosilver/-PLGA coatings can be compared to each other, the vehicle coating alone and to the current standard of care i.v. vancomycin prophylaxis used for MRSA by evaluating the following 4 groups: (1) Nanosilver/PLGA coating 1.0%; (2) Nanosilver/PLGA 2.0%; (3) PLGA vehicle coating alone (no Nanosilver); and (4) PLGA vehicle coating alone+i.v. vancomycin (100 mg/kg) at 2 h pre- and 6 h post-operatively. Overall, these data show that nanosilver selectively inhibits fibroblast proliferation over osteoblast proliferation (e.g., FIG. 13A and FIG. 13B).

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Studies on Using an Engineered Galvanic Redox System to Generate Positive Surface Potentials that Promote Osteogenic Functions

### Summary

Successful osseointegration of orthopaedic and orthodontic implants is dependent on a competition between osteogenesis and bacterial contamination on the implant-tissue interface. Previously, by taking advantage of the highly interactive capabilities of silver nanoparticles (AgNPs), we effectively introduced an antimicrobial effect to metal implant materials using a AgNP/poly(_{DL}-lactic-co-glycolic acid) (PLGA) coating. Although electrical forces have been shown to promote osteogenesis, creating practical materials and devices capable of harnessing these forces to induce bone regeneration remains challenging. Here, we applied galvanic reduction-oxidation (redox) principles to engineer a nanoscale galvanic redox system between AgNPs and 316L stainless steel alloy (316L-SA). Characterized by SEM, EDS, AFM, KPFM, and contact angle measurement, the surface properties of the yield AgNP/PLGA-coated 316L-SA (SNPSA) material presented a significantly increased positive surface potential, hydrophilicity, surface fractional polarity, and surface electron accepting/donating index. Importantly, in addition to its bactericidal property, SNPSA's surface demonstrated a novel osteogenic bioactivity by promoting peri-implant bone growth. This is the first report describing the conversion of a normally deleterious galvanic redox reaction into a biologically beneficial function on a biomedical metal material. Overall, this study details an innovative strategy to design multifunctional biomaterials using a controlled galvanic redox reaction, which has broad applications in material development and clinical practice.

### Introduction

Despite progressive advancements in bone repair devices and techniques, approximately 5.8% of metal implant failures transpire due to insufficient bone growth and osseointegration.¹ The osseointegration quality of an implant relies on its ability to promote the differentiation and incorporation of host tissue cells while inhibiting the adhesion and proliferation of bacterial cells.² Therefore, it is imperative to design orthopaedic and orthodontic implants that promote the osteogenesis of host tissue cells, but that also concurrently reduce microbial infections.³⁻⁴

To achieve this goal, processes that modify osteoinductive/osteoconductive material surface physiochemical properties, including the topography,⁵ surface chemical property,⁶ and electrical property,⁷⁻⁸ have been investigated. For instance, electrical stimulation can promote bone regeneration.⁹ Although the mechanism is not completely understood, collagen's piezoelectric property can generate a built-in electric field in the bone organic matrix,¹⁰ which may activate the membrane receptors on osteoprogenitor cells to subsequently induce osteogenesis.¹¹ Beyond this inherent property, faradic products generated around cathodic sites during electrical stimulation also appear to contribute to bone regeneration.¹² The cations, such as Ca²⁺, have the ability to rapidly deposit around the cathode, and anions, such as PO₄³⁻, HPO₄²⁻ and OH⁻, subsequently aggregate around the cations.¹³ These depositions result in the formation of hydroxyapatite at the cathode, which promotes bone formation.¹³ Attempts to induce osteogenesis with electric forces have used various methods, including direct electrical current,⁶ capacitive coupling,¹⁴ and inductive coupling.⁸ However, the requirement of external devices to generate an electrical potential, invasive procedural methods, and high infection rates have considerably halted the application of electric stimulation in clinical settings.¹⁵

It is well known that galvanic reduction-oxidation (redox) reactions occur on the surface of carbon steel in moist environments.¹⁶⁻¹⁸ In this system, iron (Fe) acts as an anode, and the numerous interstitial doped surface carbon (C) atoms act as nanoscale cathodic sites. The electron flow from the anode (Fe) to the cathode (C) leads to an increased electron density and a higher negative electric potential on the anode than on the cathodic sites.¹⁶⁻¹⁸ To harness this phenomenon, we sought to delicately engineer a similar nanoscale galvanic redox system that generates a positive surface potential (SP) on a biomedical metal material, and as a result, promotes bone growth and osseointegration of a metal implant.

Due to the large surface-to-mass ratio, silver nanoparticles (AgNPs) offer a greater active surface, higher solubility, and more chemical reactivity than non-nanoscale silver preparations. In comparison with non-nanoscale silver preparations, AgNPs have a greater release of oxidative Ag⁺ and/or more partially oxidized AgNPs with chemisorbed (surface-bound) Ag(I).¹⁹ Importantly, the electrode potential of the Ag particles significantly increases with a decrease in particle size, especially when their size is reduced to nano-scale.²⁰ In addition, the immense active surface of the spherical AgNPs is critical for their antibacterial properties. Accumulating evidence demonstrates that AgNPs are effective, broad-spectrum antimicrobial agents that can be used in a wide range of doses with a diversity of materials to prevent and manage contamination and biofilm formation without toxicity.²¹⁻²⁴ Thus, AgNPs are desirable candidates for building a galvanic redox system with antimicrobial properties. Meanwhile, our previous studies have shown that poly(_{DL}-lactic-co-glycolic acid) (PLGA) is an osteoconductive material capable of supporting a homogeneous distribution of AgNPs. PLGA is used widely with other components of conducting polymers that permit electric current to pass.²⁵ Therefore, in this study, we coated AgNPs/PLGA on a biomedical metal, 316L stainless steel alloy (316L-SA), to empower a built-in electrical force on the surface of a metal implant. The central theme of this study is: AgNPs can function similarly to doped carbon atoms in carbon steel and initiate an electron flow from the substrate metal, 316L-SA, to the cathodic AgNPs in the coated surface, as the electron transfer is driven by the difference in electrode potential between the AgNPs and 316L-SA. As a result, we expected that the controlled galvanic redox reaction of the AgNP/PLGA-coated 316L-SA (SNPSA) would create a unique surface electrical property that could be regulated by AgNP concentration to effectively stimulate local osteogenesis and osseointegration.

Although 316L-SA contains a 16-18.5% of chromium (by weight), and can form a passivation layer of chromium (III) oxide (Cr₂O₃) when exposed to oxygen, it is still more active than Ag, as shown in galvanic series charts delineating the relationships between different metals and their relative propensity to undergo redox reactions.²⁶⁻²⁷ Thus, the different electrode potentials between 316L-SA and AgNPs make the galvanic redox reactions possible. In comparison, when titanium is exposed to oxygen, it immediately forms a stable, protective titanium oxide passivation layer on its surface that imparts a noble property. In this case, the electrode potential of the titanium substrate is close to that of Ag in the galvanic series,²⁶⁻²⁷ and we inferred that there would be no such galvanic redox reaction between the AgNPs and titanium substrate. To test our theory, titanium was used as a minimally reactive substrate to fabricate AgNP/PLGA-coated titanium (SNPT).

### EXPERIMENTAL SECTION

### Materials

Spherical AgNPs (20-40 nm, QSI-Nano Silver) were purchased from QuantumSphere, Inc. (Santa Ana, Calif., United States). PLGA (lactic:glycolic=85:15, inherent viscosity: 0.64 dl/g in chloroform) was purchased from Durect Co. (Pelham, Ala., United States). Kirschner (K)-wires of 316L-SA and titanium (length: 70 mm, diameter: 0.8 mm) were purchased from Synthes, Inc. (Monument, Colo., United States), while 316L-SA and titanium discs (diameter: 7 mm) were sliced from metal rods purchased at Stainless Supply (Monroe, N.C., United States) using an electrical discharging machine at the University of California, Los Angeles (UCLA). All the other used chemicals were purchased from Sigma-Aldrich (St. Louis, Mo., United States).

### Fabrication of SNPSA and SNPT

We employed an electro-spraying method to prepare the AgNP-coated metal materials. Spherical AgNPs were dispersed into PLGA/1,4-dioxane solution and then sprayed onto the metal materials. PLGA was used because it is both biodegradable and biocompatible, and was approved by the U.S. Food and Drug Administration for clinical application. Briefly, metal K-wires and discs were fixed on a lathe mandrel and rotated at a speed of 3,450 rpm. A total of 0.25 mL AgNP/PLGA/1,4-dioxane solution was electro-sprayed onto each K-wire surface over the course of 5 min. For each disc, a total of 0.05 mL AgNP/PLGA/1,4-dioxane solution was electro-sprayed onto the surface over the course of 1 min. The coated samples were placed into an oven at a temperature of 40° C. overnight and then transferred to a fume hood for 2 days of air-drying. After drying completely, the AgNP/PLGA-coated metal materials were hermetically sealed and stored at −20° C. until their use. Electro-spraying resulted in higher AgNP proportions in the AgNP/PLGA-layer without particle aggregation. The densities of the AgNP/PLGA layer were 0.263, 0.278, and 0.293 g/cm³ at proportions of 0%, 10%, and 20% AgNP, respectively, and the densities of AgNPs in the coating surface were 0, 6.95, and 14.65 μg/cm² for 0%, 10%, and 20% AgNP/PLGA-coated metal materials, correspondingly.

### Scanning Electron Microscopy (SEM) and Energy-Dispersive X-Ray Spectroscopy (EDS)

SEM (Nova NanoSEM 230-D9064, FEI Company, Hillsboro, Oreg., United States) was used to evaluate the morphology of AgNP/PLGA-coated metal materials, while EDS was documented simultaneously.^{21,28} The surface atomic composition of silver (SACₛ) was analyzed based on the EDS measurement. The testing parameters were set to WD: 15 mm, primary electron energy: 10 keV, and process time: 5 s. For the EDS measurements, five samples were scanned for each group. Three different 80×40 μm areas were selected from each sample. Each area was scanned in quintuplicate.

### Atomic Force Microscopy (AFM) and Kelvin Probe Force Microscopy (KPFM)

The surface roughness (Ra; the arithmetic average of the absolute roughness profile values) of AgNP/PLGA-coated metal materials was assessed by topographic AFM imaging using the Bruker Dimension Icon Scanning Probe Microscope (Bruker Nano, Inc., Santa Barbara, Calif., United States) in ambient conditions. Tapping (AM-AFM) mode imaging employed silicon cantilever probes (RTESP, Bruker Nano, Inc.) with nominal tip radii of 8 nm, spring constants of approximately 30 N/m, and resonant frequencies of 260-325 kHz. Height and phase images (2×2 μm) were acquired simultaneously using a 1 Hz scan rate. An automatic algorithm was used to flatten the images. Ra was quantified using the NanoScope Analysis V1.40 software package (Bruker Nano, Inc.).

Localized SPs of SNPSA and SNPT were characterized by KPFM. KPFM imaging was conducted in the dual-pass amplitude modulated lift mode using Pt-Ir coated silicon probes (SCM-PIT, Bruker Nano, Inc.) with nominal tip radii of 20 nm, spring constants of approximately 3 N/m, and resonant frequencies of 60-80 kHz. Co-localized topographic and SP images were acquired over 25×25 μm regions at a lift height of 100 nm. Reported values refer to the contact potential difference between the Pt—Ir tip and surface. To minimize measurement variability, a single KPFM probe was used in the comparisons between SNPSA and SNPT, and five different locations on each sample surface from five samples in each group were analyzed.

### Wettability and Surface Free Energy Characterization

Wettability and surface free energy values were obtained from contact angle (θ) measurements.²⁸⁻³¹ Advancing contact angles of multiple standard liquids (water-miscible dipolar liquids: formamide and ethylene glycol; water-immiscible non-polar liquid: diiodomethane) on the tested AgNP/PLGA-coated metal materials were measured using a contact angle analyzer (FPA125; First Ten Angstroms, Portsmouth, Va., United States). The surface tension properties of these standard liquids were listed in Table 7.

TABLE 7
Surface tension properties of standard liquids
used in this study at 20° C. in mJ/m^{2 1}

| Liquid | γ_{L} | γ_{L}^{LW} | γ_{L}^{AB} | γ_{L}⁺ | γ_{L}⁻ |
|---|---|---|---|---|---|
| Formamide | 58.0 | 39.0 | 19.0 | 2.28 | 39.6 |
| Ethylene glycol | 48.0 | 29.0 | 19.0 | 3.0 | 30.1 |
| Diiodomethane | 50.8 | 50.8 | 0 | 0.01 | 0 |

Note:
All the standard liquids used in this study were purchased from Sigma-Aldrich (St. Louis, MI). γ_{L}, γ_{L}^{LW}, γ_{L}^{AB}, γ_{L}⁺, γ_{L}⁻ represent surface tension, non-polar Lifshiz-van der Waals component, polar Lewis acid-base component, Lewis acid component, and Lewis base component of standard liquids, respectively.

The surface tension components of these liquids were analyzed based on the measured contact angles. Based on the Derjaguin, Landau, Vervey, and Overbeek (DLVO) model, the solid surface free energy (γ_{S}) can be divided into a non-polar Lifshiz-van der Waals component (γ_{S}^{LW}) and a polar Lewis acid-base component (γ_{S}^{AB}), which is expressed as the geometric mean of the Lewis acid component electron acceptor) and Lewis base component (γ_{S}⁻, electron donor).³⁰ For a solid surface, the process can be described by Eq. 1. For solid/liquid interfacial interaction, γ_{S}, γ_{S}^{LW}, γ_{S}^{AB}, γ_{S}⁺, and γ_{S}⁻ can be calculated according to Eq. 2.³⁰⁻³¹ In addition, the surface fractional polarity (SFP) was determined by γ_{S}^{AB}/γ_{S}, and the surface electron accepting/donating index (SEADI) was defined as the ratio of the electron-accepting parameter [(γ_{S}⁺)^{1/2}] and electron-donating parameter [(γ_{S}⁻)^{1/2}]. Five samples were tested for each group.

γ_{S}=γ_{S}^{LW}+γ_{S}^{AB}=γ_{S}^{LW}+2·(γ_{S}^{+·γ}_{S}⁻)^{1/2} (Eq. 1)

γ_{L}·(1+cos θ)=2·(γ_{S}^{LW}·γ_{L}^{LW})^{1/2}+2·(γ_{S}⁺·γ_{L}⁺)^{1/2}+2·(γ_{S}⁻·γ_{L}⁻)^{1/2} (Eq. 2)

where γ_{L}, γ_{L}^{LW}, γ_{L}⁺ and γ_{L}⁻, represent surface tension, non-polar Lifshiz-van der Waals component, Lewis acid component, and Lewis base component of standard liquids, respectively.

### Conditional Osteogenic Medium (COM) Treatment

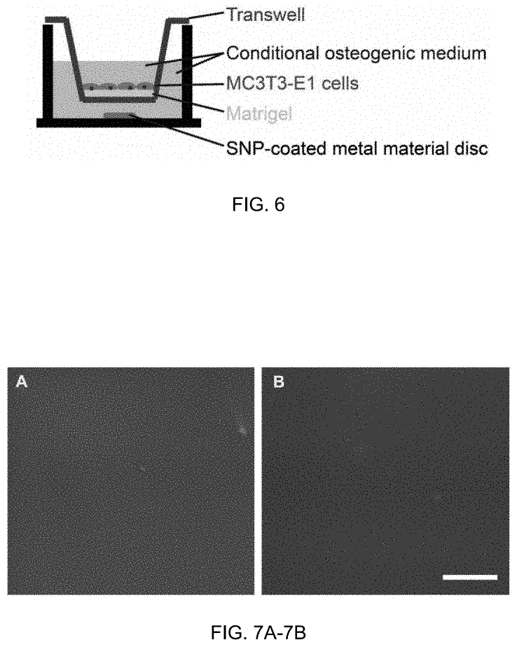

Due to the dynamic and irreversible changes that surface properties of a material undergo during a successive osteogenesis process,³² we used a COM treatment to mimic the physiology in which the biological and non-biological components meet and interact on the implant surface. Transwell® inserts were used to separate the cells and the coating surface during incubation (FIG. 6) to preserve the physicochemical properties of the coating surface after COM treatment and eliminate damage to the coating matrix during the cell removal process mediated by trypsin digestion and mechanical scratching. Briefly, SNPSA and SNPT discs were incubated with 500 μl of osteogenic medium (a-minimum essential media supplied with 10% fetal bovine serum, 1% HT supplement, 100 unit/ml penicillin, 100 μg/ml streptomycin, 50 μg/ml ascorbic acid, and 10 mM β-glycerophosphate) at 37° C. for 6 days. To avoid the influence of direct cellular contact on surface morphology, 2×10³ pre-osteoblastic MC3T3-E1 cells (subclone 4, ATCC® CRL-2593; Manassas, Va., United States) were cultured on Matrigel® (BD Biosciences, San Jose, Calif., United States) pre-coated Transwell® plates (Corning Inc., Corning, N.Y., United States).

### MC3T3-E1 Cell Proliferation and Osteogenic Differentiation

MC3T3-E1 cells were seeded on SNPSA and SNPT metal discs at a density of 2×10³ cells per disc and cultivated in the osteogenic medium in 24-well cell culture plates at 37° C. Cell proliferation on the AgNP/PLGA-coated metal discs was evaluated by the Vybrand® MTT Cell Proliferation Assay Kit (Thermal Fisher Scientific, Canoga Park, Calif., United States) after 9 days of cultivation. Alkaline phosphatase (ALP) activity, assessed by the 1-Step™ NBT/BCIP Substrate Solution (Thermal Fisher Scientific) at day 9, and the degree of mineralization, assessed by Alizarin Complexone staining (Thermal Fisher Scientific) at day 21, were used to quantify cellular differentiation. Images were taken by a fluorescence microscope (Olympus BX51, Tokyo). The mineralized area was defined as [(staining area/total disc area)×100] (%) using Image J software.

After 6 days of cultivation, total RNA or total protein was isolated by the RNeasy Mini Kit with DNase treatment (Qiagen, Valencia, Calif., United States) or RIPA Buffer (Pierce Biotechnology, Rockford, Ill., United States). One μg total RNA was used for reverse transcription with the iScript™ Reverse Transcription Supermix for quantitative real-time PCR (qRT-PCR) (Bio-Rad Laboratories, Hercules, Calif., United States). qRT-PCR was performed with TaqMan® Gene Expression Assays (Life Technologies) and SsoFast™ Probes Supermix with ROX (Bio-Rad Laboratories) on a 7300 Real-Time PCR system (Applied Biosystems Inc, Foster City, Calif., United States). Osteogenic growth factors, such as transforming growth factor (Tgf)β1, bone morphogenetic protein (Bmp)2, and Bmp4, were analyzed for osteogenesis. Concomitant glyceraldehyde 3-phosphate dehydrogenase (Gapdh) was used as a housekeeping standard. Data analysis was achieved using the _{ΔΔ}C_{T} method. Western blot analysis was performed to quantify the corresponding protein amounts. Anti-BMP2 (Abcam, Cambridge, Mass., United States), anti-BMP4 (Abcam), anti-TGFβ1 (Santa Cruz Biotechnology, Santa Cruz, Calif., United States), and GAPDH (Santa Cruz Biotechnology) primary antibodies were used at a dilution of 1:1,000. All the experiments were repeated in triplicate.

### Animal Model for In Vivo Bone Regeneration

All surgical procedures were approved by the UCLA Office of Animal Research Oversight (protocol #2012-120). A femoral intramedullary rod (FIR) model was used to assess the osteogenic ability of the metal materials that were utilized as intramedullary fixation devices in vivo.³³ 12-week old male Sprague-Dawley rats were randomly assigned to groups with different types of K-wire implants, with 5 rats in each treatment group. Briefly, the rats were anesthetized by isoflurane inhalation, the left femur was aseptically prepared, and an approach to the distal femur was made via a lateral knee arthrotomy. A 20-gauge needle was used to create an entry port into the proximal aspect of the femur medullary canal in order to ream the canal in preparation for placement of the intramedullary rod. A coated K-wire (2.7 cm in length) was inserted with the narrow portion first entering into the medullary canal, and then seated into the cortical bone in the distal aspect of the femur. The overlying muscle and fascia were closed with a 4-0 Vicryl absorbable suture. Following surgery, the animals were housed in separate cages and allowed to eat and drink ad libitum. Weight-bearing began immediately postoperatively, and the animals were monitored daily. Buprenorphine was administered for 2 days as an analgesic, but no antibiotics were administered post-surgery. The rats were euthanized by CO₂ treatment at 8 weeks post-implantation. No animals were excluded from the analysis.

### 3D Micro-Computed Tomography (μCT) Scanning

Animals were euthanized at 8 weeks post-implantation. FIR model femurs were harvested and fixed in 4% paraformaldehyde for 48 hours. The samples were scanned using high-resolution μCT (SkyScan 1176, Bruker micro-CT N.V., Kontich, Belgium) at an image resolution of 18.0 μm (90 kV and 278 μA radiation sources with a 0.1 mm aluminum filter). 3D high-resolution images were generated by the CTAn software, following instructions provided by the manufacturer, and improved by Blender software. The ratio of bone volume (BV) to total volume (TV) was used to quantify bone tissue generation in vivo.

### Histological Analysis

After μCT scanning, specimens were dehydrated with a graded solution of ethanol and cleared with xylenes. Specimens were then embedded in a fresh solution of methyl methacrylate, dibutyl phthalate, and Perkadox-16, and subsequently underwent polymerization. Specimens were cut as consecutive slides using Donath's technique and the EXAKT Cutting and Grinding System (EXAKT Technologies, Oklahoma City, Okla., United States), and stained with Sanderson's Rapid Bone Stain. Van Gieson-Picrofuschsin was used as a counterstain. Specimens were imaged using an Olympus BX51 microscope. The area of mineralized bone was further quantified by Image J software.

### Statistical Analysis

All statistical analyses were conducted in consultation with the UCLA Statistical Biomathematical Consulting Clinic. Statistical analyses were computed by OriginPro 8 (Origin Lab Corp., Northampton, Mass., United States). Data are generally presented as mean±the standard deviation and compared by one-way ANOVA and two-sample t-tests Mann-Whitney and Kruskal-Wallis ANOVA tests were used for non-parametric data. A Pearson's correlation coefficient was used for correlation tests. The p-values less than 0.05 were considered statistically significant.

### Results

### Fabricating and Characterizing the Surfaces of SNPSA and SNPT Materials

The SNPSA and SNPT materials were fabricated by the same electro-spraying method, and their graphical structures were illustrated in FIG. 1A-1C. Employing electrochemical principles, we hypothesized that a nanoscale structure capable of enabling a galvanic redox reaction could be established on the SNPSA materials. The AgNPs embedded in the AgNP/PLGA matrix served as cathodic sites in the presence of moisture (FIG. 1A, FIG. 1B) and 316L-SA was oxidized and served as an anode in the galvanic redox system (FIG. 1A, FIG. 1B). Due to the noble metal property of the passive oxidized titanium surface, the titanium substrate and AgNPs cannot undergo redox reactions on the AgNP/PLGA-coated titanium (SNPT), even when the AgNP/PLGA-coatings of SNPT and SNPSA have the same composition and morphology (FIG. 1C).

FIG. 1A-1C mechanism illustrations as follows: (A). The positive surface potential of the AgNP/PLGA-coated 316L-SA (SNPSA) was generated by galvanic redox reactions, in which the iron (Fe) in 316L-SA was oxidized to Fe²⁺, and the released electrons (e) were transferred to the AgNPs cathodes. Meanwhile, the H⁺, Ag⁺, and O₂ were reduced at SNPSA's cathodic sites. A positive surface potential and corresponding electric field were established around the cathodic sites. (B). The electron flow positively correlated with the AgNP proportion in the PLGA layer. In comparison to the 10% AgNP surfaces, the 20% AgNP surfaces had more AgNPs that were connected together to form electron conducting paths. This lead to a greater electron flow and resulted in both higher surface potential and osteogenic ability. (C). There was no electron transfer from titanium to AgNP surface due to the noble metal property of the titanium surface, thus no nanoscale galvanic redox reactions occurred on the SNPT material.

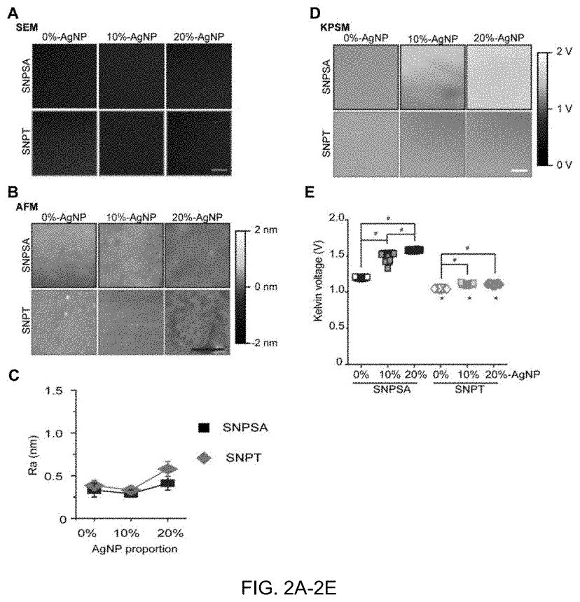

To test our hypothesis that the positive SP depends on the AgNP proportion in the PLGA layer, AgNP/PLGA matrices with different proportions of AgNPs were electro-sprayed onto the 316L-SA and titanium materials. EDS analysis identified Ag as an elemental component on the material surfaces (Table 2), which provides direct evidence that AgNPs were incorporated into the surfaces of the AgNP/PLGA-coated metal materials. SEM and AFM analyses revealed an even and smooth AgNP/PLGA layer on the 316L-SA and titanium without any distinct morphological differences (FIG. 2A, FIG. 2B). This was further confirmed by Ra quantification (FIG. 2C). The SP of the coating was analyzed by KPFM, which revealed the electronic homogeneity of the measured surface potentials of SNPSA and SNPT (FIG. 2D). As we hypothesized, SNPSA exhibited significantly higher SP values when compared with the control (0% SNPSA without any encapsulated AgNPs), and the SP values are proportional to the AgNP content in the coating layer (FIG. 2E). For example, the SP of 20%-SNPSA was 0.5 mV more positive than that of the 0%-SNPSA, while SNPT's corresponding SP value increase was less than 0.1 mV. Additionally, the SNPT samples that had the same AgNP proportions as the SNPSA samples retained lower SP values than the SNPSA counterparts (FIG. 2E). These results demonstrate that the SP of AgNP/PLGA-coated metal material was dependent on the AgNP proportion in the AgNP/PLGA matrix, as well as the electrode potential of the metal substrates used.

TABLE 2
EDS determination of SNPSA and
SNPT surface atomic compositions.

| | AgNP | | | |
|---|---|---|---|---|
| Metal | proportion | O atom | C atom | Ag atom |
| material | (%) | (%)* | (%)* | (%)* |
| SNPSA | 0 | 38.7 ± 0.4 | 61.3 ± 0.4 | — |
| | 10 | 38.1 ± 0.1 | 59.3 ± 0.1 | 2.6 ± 0.1 |
| | 20 | 36.8 ± 0.4 | 60.4 ± 0.5 | 2.8 ± 0.1 |
| SNPT | 0 | 38.6 ± 0.2 | 61.4 ± 0.2 | — |
| | 10 | 37.9 ± 0.6 | 59.8 ± 0.9 | 2.3 ± 0.4 |
| | 20 | 36.6 ± 0.1 | 60.8 ± 0.2 | 2.6 ± 0.2 |

*Data are shown as the mean ± standard deviation (n = 10).

FIG. 2A-2E show SNPSA and SNPT surface morphologies and surface potentials: (A) Scanning electron microscopy (SEM) demonstrated an even, smooth AgNP/PLGA coating on SNPSA and SNPT with increasing AgNP proportions (0%, 10%, 20%) in the coating layer. Scale bar: 20 μm. (B) Atomic force microscopy (AFM) images confirmed that a homogeneous AgNP/PLGA matrix layer was generated on both the 316L-SA and titanium materials. Scale bar: 1 μm. (C) Measurements of surface roughness (Ra) show that there were no statistical differences between SNPSA and SNPT materials with the same AgNP proportions (N=3). (D) Kelvin probe force microscopy (KPFM) documented the surface potentials (SP) of SNPSA and SNPT. Scale bar: 5 μm. (E) The SP was quantified by analyzing a variety of different locations (N=29). SNPSA had an increased SP, while no significant change in SNPT's SP was detected, even with an increasing AgNP proportion Mann-Whitney analyses were used to detect statistical differences. # (p<0.05), significant difference resulting from different AgNP proportions; * (p<0.05), significant difference between SNPT and SNPSA with the same AgNP proportions.

The surface hydrophobic/hydrophilic properties of SNPSA and SNPT were compared by a contact angle measurement. The results showed that the surface contact angles of water-miscible dipolar liquids, formamide and ethylene glycol, were significantly decreased on both SNPSA and SNPT with increasing AgNP proportions in the AgNP/PLGA matrix, while the contact angles of water-immiscible non-polar diiodomethane were slightly increased (Table 3). These findings indicate that AgNP incorporation contributed to the hydrophilicity of the AgNP/PLGA-coated metal material surfaces. More importantly, the surface contact angles of water-miscible dipolar liquids on SNPSA were much smaller than the angles on SNPT when the same AgNP proportion was present in both of the respective surfaces (Table 3).

TABLE 3
The surface contact angles of SNPSA and SNPT at 20° C.

| Coated | AgNP | | | |
|---|---|---|---|---|
| metal | proportion | Contact angle (θ) ^{*} | | |
| materials | (%) | Formamide | Ethylene glycol | Diiodomethane |
| SNPSA | 0 | 49.1 ± 0.3 | 42.9 ± 0.4 | 43.6 ± 0.2 |
| | 10 | 31.6 ± 0.3 | 18.3 ± 0.4 | 44.7 ± 0.1 |
| | 20 | 28.9 ± 0.2 | 11.5 ± 0.2 | 45.8 ± 0.4 |
| SNPT | 0 | 49.8 ± 0.4 | 43.7 ± 0.1 | 43.5 ± 0.1 |
| | 10 | 36.9 ± 0.1 | 27.6 ± 0.2 | 44.2 ± 0.2 |
| | 20 | 34.3 ± 0.1 | 23.7 ± 0.3 | 45.0 ± 0.2 |

^{*} Data are shown as the mean ± standard deviation (n = 6).

The calculated γ_{S}, γ_{S}^{LW}, γ_{S}^{AB}, and γ_{S}⁻ of AgNP/PLGA-coated metal materials are summarized in Table 4. As expected, 0%-SNPSA and 0%-SNPT surfaces presented similar surface free energy values; however, the incorporation of AgNPs significantly increased γ_{S}^{AB}, which resulted in a higher SFP and SEADI in the AgNP/PLGA-coated metal materials, especially for the SNPSA (Table 4). These findings indicate that AgNPs play an important role in the proposed galvanic system. The SEADI of SNPSA was much higher than that of SNPT, which suggests that the SNPSA surface undergoes active electron transfer attributed to the electrochemical redox reaction. Moreover, the SEADI and SFP of SNPSA materials became significantly larger with increasing AgNP proportion, but the corresponding SNPT materials only experienced a limited increase in SEADI and SFP with increasing AgNP proportions (Table 4). These data strongly support our hypothesis that the nanoscale galvanic redox system is established on SNPSA, but not the surface of SNPT, as shown in FIG. 1A-1C.

TABLE 4
Surface free energy components of SNPSA and SNPT at 20° C.

| | | | | | | | SFP | SEADI |
|---|---|---|---|---|---|---|---|---|
| Metal material | AgNP proportion (%) | γ_{S} | γ_{S}^{LW} | γ_{S}^{AB} | γ_{S}⁺ | γ_{S}⁻ | $( \frac{{γ}_{S}^{AB}}{{γ}_{S}} \% )$ | $\frac{{( {γ}_{S}^{+} )}^{1 \text{/} 2}}{{( {γ}_{S}^{-} )}^{1 \text{/} 2}}$ |
| SNPSA | 0 | 38.48 | 37.80 | 0.68 | 0.05 | 2.19 | 1.77 | 0.151 |
| | 10 | 40.78 | 37.06 | 3.72 | 0.66 | 5.28 | 9.13 | 0.354 |
| | 20 | 41.74 | 36.41 | 5.33 | 1.25 | 5.69 | 12.8 | 0.469 |
| SNPT | 0 | 38.45 | 37.78 | 0.67 | 0.05 | 2.04 | 1.74 | 0.157 |
| | 10 | 38.86 | 37.36 | 1.50 | 0.12 | 4.58 | 3.86 | 0.162 |
| | 20 | 38.84 | 36.94 | 1.91 | 0.17 | 5.22 | 4.92 | 0.180 |

Notes:
γ_{S}: solid surface free energy component;
γ_{S}^{LW}: non-polar Lifshiz-van der Waals component:
γ_{S}^{AB}: polar Lewis acid-base component;
γ_{S}⁺: Lewis acid component, electron acceptor;
γ_{S}⁻: Lewis basic component, electron donor;
SFP: surface fractional polarity;
SEADI: surface electron accepting/donating index.

### Surface Property Change After COM Treatment

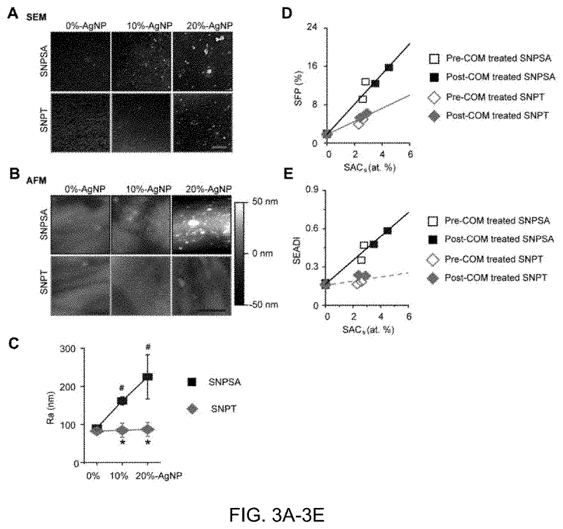

After COM treatment, the sample surface was characterized by SEM, EDS, AFM, and contact angle measurement. Although SNPSA and SNPT presented similarly smooth surface morphologies pre-COM treatment (FIG. 2A-2C), the SNPSA surfaces had markedly more heterogeneous morphologies post-COM treatment, which is consistent with the increased Ra values that were obtained (FIG. 3A-3C). SNPSA had significantly higher Ra values than SNPT post-COM treatment (FIG. 3C). Additionally, the surface free energy (calculated based on contact angles in Table 5) indicates that the AgNP incorporation in the coating layer of the SNPSA materials led to significant increases in γ_{S}^{AB}, SFP, and SEADI values (Table 6). These results also showed that the SACₛ was significantly increased post-COM treatment on the surface of SNPSA, but not on the surface of SNPT (FIG. 3D, FIG. 3E). This suggests that more AgNPs were exposed on the surface of the SNPSA material post-COM treatment, and thus, the polar γ_{S}^{AB} was much higher. This also increased the total γ_{S} and SFP values. A linear relationship between SFP and SACₛ was observed in both SNPSA and SNPT (FIG. 3D, SNPSA: SFP=3.47×SACₛ+1.66, Pearson's correlation coefficient=0.964; SNPT: SFP=1.11×SACₛ+1.70, Pearson's correlation coefficient=0.974). The SNPSA had a much higher correlation slope than SNPT, which indicates that the SFP of SNPSA was more sensitive to the AgNP proportion in the AgNP/PLGA matrix. Importantly, the SEADI of SNPSA was linearly correlated to its SACₛ, but the SEADI of SNPT was not linearly correlated with its SACₛ (FIG. 3E, SNPSA: SEADI=0.0984×SACₛ+0.148, Pearson's correlation coefficient=0.955; SNPT: SEADI=0.0063×SACₛ+0.156, Pearson's correlation coefficient=0.743).

TABLE 5
The surface contact angles of SNPSA and
SNPT after COM treatment at 20° C.

| Coated | AgNP | | | |
|---|---|---|---|---|
| metal | proportion | Contact angle (θ) ^{*} | | |
| materials | (%) | Formamide | Ethylene glycol | Diiodomethane |
| SNPSA | 0 | 49.2 ± 0.5 | 43.0 ±0.6 | 44.2 ± 0.6 |
| | 10 | 30.8 ± 0.4 | 15.7 ± 0.3 | 46.0 ± 0.5 |
| | 20 | 27.4 ± 0.5 | 3.6 ± 0.6 | 46.3 ± 0.5 |
| SNPT | 0 | 48.9 ± 0.3 | 42.7 ± 0.4 | 44.3 ± 0.4 |
| | 10 | 38.0 ± 0.4 | 28.7 ± 0.5 | 44.9 ± 0.2 |
| | 20 | 33.3 ± 0.6 | 21.8 ± 0.5 | 45.2 ± 0.3 |

^{*} Data are shown as the mean ± standard deviation (n = 6).

TABLE 6
Surface free energy components of SNPSA and SNPT after COM treatment at 20° C.

| | | | | | | | SFP | SEADI |
|---|---|---|---|---|---|---|---|---|
| Metal material | AgNP proportion (%) | γ_{S} | γ_{S}^{LW} | γ_{S}^{AB} | γ_{S}⁺ | γ_{S}⁻ | $( \frac{{γ}_{S}^{AB}}{{γ}_{S}} \% )$ | $\frac{{( {γ}_{S}^{+} )}^{1 \text{/} 2}}{{( {γ}_{S}^{-} )}^{1 \text{/} 2}}$ |
| SNPSA | 0 | 38.16 | 37.38 | 0.78 | 0.07 | 2.22 | 2.04 | 0.178 |
| | 10 | 41.44 | 36.30 | 5.14 | 1.22 | 5.39 | 12.4 | 0.476 |
| | 20 | 42.83 | 36.09 | 6.74 | 1.97 | 5.76 | 15.7 | 0.585 |
| SNPT | 0 | 38.05 | 37.32 | 0.73 | 0.06 | 2.30 | 1.92 | 0.162 |
| | 10 | 39.03 | 36.97 | 2.06 | 0.24 | 4.36 | 5.28 | 0.235 |
| | 20 | 39.27 | 36.82 | 2.45 | 0.28 | 5.35 | 6.24 | 0.229 |

Notes:
γ_{S}: solid surface free energy component;
γ_{S}^{LW}: non-polar Lifshiz-van der Waals component;
γ_{S}^{AB}: polar Lewis acid-base component;
γ_{S}⁺: Lewis acid component, electron acceptor;
γ_{S}⁻: Lewis basic component, electron donor;
SFP: surface fractional polarity;
SEADI: surface electron accepting/donating index.

FIG. 3A-3E show surface morphologies and properties of SNPSA and SNPT after COM treatment: (A) & (B) 6 days after COM treatment, SEM and AFM images showed that the SNPSA surfaces presented markedly heterogeneous morphologies with increasing AgNP proportions (0%, 10%, 20%), while the SNPT surfaces did not show a significant change post-COM treatment. Scale bar in a: 20 μm. Scale bar in b: 1 μm. (C) Surface roughness (Ra) measurement showed a significant difference in the surface morphology between SNPSA and SNPT at various AgNP proportions (0, 10%, 20%) post-COM treatment. (D) A linear relationship between surface fractional polarity (SFP) and surface atomic composition of silver (SACₛ) was observed in both SNPSA and SNPT at various AgNP proportions (0, 10%, 20%) (SNPSA: SFP=3.47×SACₛ+1.66, Pearson's correlation coefficient=0.964; SNPT: SFP=1.11×SACₛ+1.70, Pearson's correlation coefficient=0.974). (E). A linear relationship between surface electron accepting/donating index (SEADI) and SACₛ was observed in SNPSA (0%, 10%, 20%) (SEADI=0.0984×SACₛ+0.148, Pearson's correlation coefficient=0.955), but not between the SEADI and SACₛ of SNPT (0%, 10%, 20%) (SEADI=0.0063×SACₛ+0.156, Pearson's correlation coefficient=0.743). SNPSA's higher slope suggests that the Ag content on the surface had a significant effect on SNPSA's surface property due to the galvanic redox system in the coating layer. One-way ANOVA and two sample t-tests were used to detect statistical differences (N=3). # (p<0.05), significant difference in comparison with 0%-SNPSA; * (p<0.05), significant difference between SNPT and SNPSA with the same AgNP proportions.

### Evaluating the Osteogenic Activities of SNPSA and SNPT In Vitro

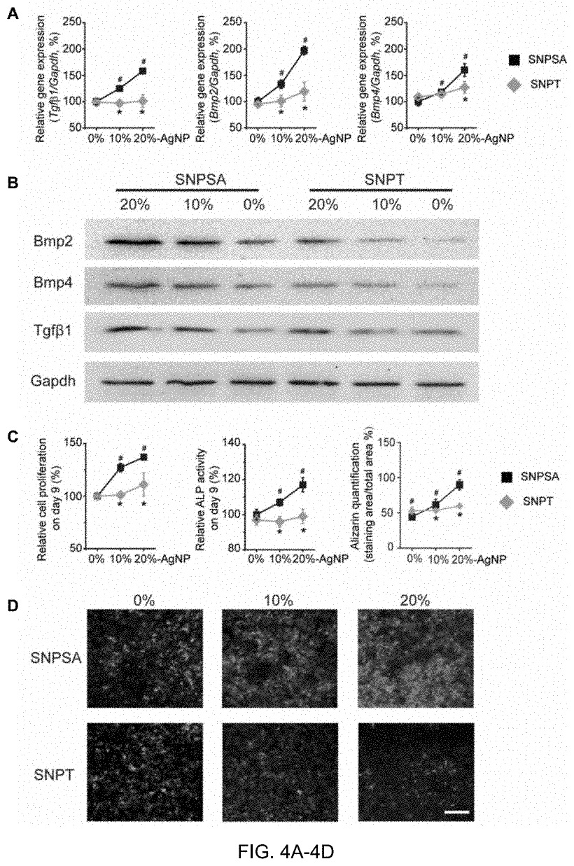

To determine whether the surface potential generated on SNPSA surface can promote osteogenic differentiation in vitro, pre-osteoblastic MC3T3-E1 cells were cultured on SNPSA and SNPT discs. Previously, electric fields have been reported to induce the expression of osteogenic growth factors, including TGFβ1, BMP2, and BMP4, in osteoblastic cells.³⁴⁻³⁵ In this study, after 6 days of cell cultivation, Tgfβ1, Bmp2, and Bmp4 transcription levels of MC3T3-E1 cells grown on SNPSA were significantly increased in an AgNP-proportion-dependent manner, but there were no significant differences among MC3T3-E1 cells grown on SNPT (FIG. 4A), as confirmed by the protein expression analysis (FIG. 4B). Corresponding with the findings in our previous report,²⁸ the proliferation, ALP activity, and mineralization degree of MC3T3-E1 cells on SNPSA increased with increasing AgNP proportion (FIG. 4C, FIG. 4D). In contrast, AgNP incorporation did not affect proliferation or osteogenic differentiation of MC3T3-E1 cells seeded on SNPT. These results confirm that the SNPSA, with a positive surface potential on the AgNP/PLGA-coating layer, can induce MC3T3-E1 cell differentiation and maturation to achieve osteogenesis.

FIG. 4A-4D show osteogenic ability of SNPSA and SNPT in vitro with different AgNP proportions (0%,10%, 20%): (a) After 6 days of cultivation, MC3T3-E1 cells grown on SNPSA had significantly increased transcription levels of Tgfβ1, Bmp2, Bmp4, and Gapdh in an AgNP-proportion-dependent manner. (b) The corresponding protein amounts were determined by Western blot. (c) The growth and osteogenic differentiation of MC3T3-E1 cells on SNPSA and SNPT were determined by cell proliferation (day 9), alkaline phosphatase (ALP) activity (day 9), and terminal mineralization (day 21). (d) MC3T3-E1 cells with Alizarin Complexone staining at day 21. Scale bar: 100 μm. Data were normalized to 0%-SNPSA [N=3 (a & d) or 6 (c)]. Kruskal Wallis and Mann-Whitney analyses were used to detect statistical differences. #(p<0.05), significant difference in comparison with 0%-SNPSA; * (p<0.05), significant difference between SNPT and SNPSA with the same AgNP proportions.

### Evaluating the Bone Formation Capability of SNPSA and SNPT In Vivo

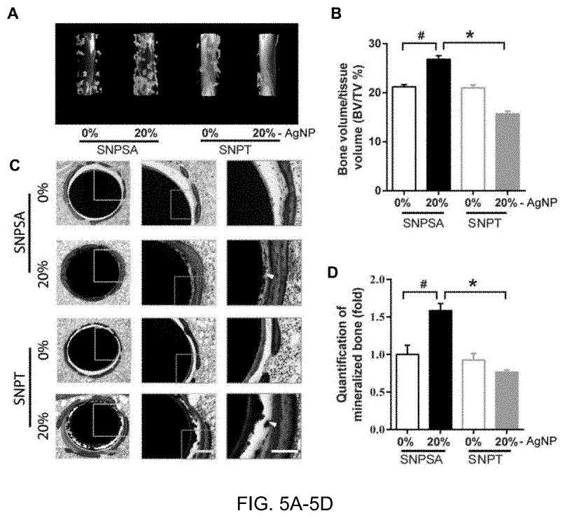

To confirm whether the surface potential generated on SNPSA could promote bone formation in vivo, SNPSA and SNPT implants were inserted into the femurs of rats to create a FIR model.³³ 3D μCT demonstrated that, after 8 weeks of implantation in the rat distal femoral cavity, more bone formed around 20%-SNPSA implants than around non-Ag coated metal materials and 20%-SNPT implants (FIG. 5A). Also, the BV/TV ratio was significantly higher in the 20%-SNPSA implants than that of the other tested implants (FIG. 5B). However, there were no significant differences in the μCT images and BV/TV ratios between the 0%-SNPSA and 0%-SNPT implants. Histological evaluation permitted visualization of the mineralized bone with red coloring, and soft tissue with blue coloring. Consistent with the μCT results, there was more bone formation around the 20%-SNPSA implants than around the 20%-SNPT implants FIG. 5C, FIG. 5D). Interestingly, fibrotic soft tissues (blue staining) and cartilage-like tissues (purple staining) were only observed around the 20%-SNPT implants (FIG. 5C). These results suggest that SNPSA acquired through a galvanic redox system promoted bone formation in vitro and in vivo.

Histological analysis of 0%-SNPSA and 0%, 20%-SNPT showed a gap (a region of no staining) between the new bone and the implants due to the residual AgNP/PLGA layer, but almost no gap was observed in the 20%-SNPSA implants (FIG. 5C). This demonstrates that 20%-SNPSA achieved earlier and more direct bone apposition on the implant surface compared to the other implants, which indicates a better capability for in vivo osseointegration. This phenomenon also demonstrated that the cathodic sites on the AgNP/PLGA layer of the 20%-SNPSA implants degraded faster due to the galvanic redox system, which is consistent with the finding that the SNPSA surface showed a significantly increased Ra post-COM treatment in vitro. However, since the unique AgNP/PLGA matrix structure is a prerequisite for the galvanic redox system's formation and function, the galvanic redox reaction terminates when the AgNP/PLGA matrix is degraded. Thus, the transient existence of the engineered galvanic redox system does not lead to elevated corrosion of the 316L-SA. This was evidenced in the SEM images of the surface of the 316L-SA substrate from the 20%-SNPSA implants after 8 weeks of implantation (FIG. 7A-7B). FIG. 7A-7B are SEM images of the 316L-SA surface before (A) and after (B) in vivo implantation. After 8 weeks of implantation, the SNPSA materials were taken out of the rat distal femoral cavity. The tissues and residual AgNP/PLGA layers were completely removed for observation under SEM. No visible changes in the metal surface morphology were observed after implantation. Scale bar: 20 μm.

FIG. 5A-5D show in vivo osteogenic effects of SNPSA and SNPT in a rat femoral intramedullary rod (FIR) model: (A) 3D μCT reconstruction images of new bone formation in rat FIR cavities around 0%- and 20%-SNPSA and SNPT 8 weeks post-implantation. More bone formed around 20%-SNPSA than other tested materials. (B) The ratios of bone volume to total volume (BV/TV) between SNPSA and SNPT were quantified. 20%-SNPSA with the galvanic redox reaction, showed significantly higher BV/TV when compared to the other groups. (C) Histological cross-section images of SNPSA and SNPT implants stained by Sanderson's rapid bone staining showed more mineralized bone (red staining) around the 20%-SNPSA implants. More fibrotic soft tissue (blue staining) and cartilage-like tissue (purple staining) were observed around the 20%-SNPT implants. Yellow arrows indicate the AgNP aggregation (black dots). Scale bar: 400 μm (red), 200 μm (orange), 100 μm (white). (D) Quantification analysis of the SNPSA and SNPT implant histological images. Kruskal Wallis and Mann-Whitney analyses were used to detect statistical differences. # (p<0.05), significant difference in comparison with 0%-SNPSA; * (p<0.05), significant difference between SNPT and SNPSA at the same AgNP proportions.

### Discussion

In this study, we documented a novel bioengineering strategy that generates built-in electrical forces on metal materials, which can facilitate the osteogenesis and osseointegration in vitro and in vivo. In this strategy, nano-sized silver particles were embedded into a PLGA matrix and coated onto the metal surface. Due to the different electrode potentials between the substrate metal and AgNPs, the coating matrix modified the surface electron density and surface potential, and by doing so, altered the electrochemical property of the implant surface. This is the first report that describes the employment of a galvanic redox mechanism and nanotechnology to modify a metal surface, which introduces a novel bioactivity to a metal implant typically used for structural support.

Of the elements in the engineered redox pair on the surface of SNPSA implants, Fe, the major element (>62%) of 316L-SA, can be oxidized to Fe²⁺ and release electrons that are transferred to the AgNP cathodes on the coating surface (FIG. 1A). Meanwhile, Ag ions [Ag⁺, or Ag(I)] can be reduced to Ag[Ag(0)] by accepting the electrons. AgNPs that connect to one another in the AgNP/PLGA matrix can serve as the electron conduction path between the anode and cathode sites. It should also be noted that carbon dioxide (CO₂) found in moisture (H₂O) can dissociate into bicarbonate (HCO₃⁻) and hydrogen (H⁺) ions. The H⁺ can also be generated by the degradation of PLGA. During redox reactions, H⁺ can be reduced to H₂ on the AgNP cathodes in the AgNP/PLGA-coating layer of the SNPSA materials. The electrode reactions can occur according to the equations below (Eq. 3-5):

Fe−2e⁻→Fe²⁺ (Eq. 3)

Ag⁺+e⁻→Ag (Eq. 4)

2H⁺+2e⁻H₂ (Eq. 5)

The high SPs found on the SNPSA surfaces by KPFM (FIG. 2A-2E) supported our hypothesis that the nanoscale galvanic redox reactions occurred on the surface of SNPSA. In the nanoscale galvanic redox system fabricated to create SNPSA, the cathodic AgNP/PLGA layer showed a positive SP that was dependent on the proportion of AgNPs in the PLGA layer (FIG. 2E). In contrast, the SNPT samples were not likely to develop the galvanic redox system because the electrode potential of the passive oxidized titanium surface is close to that of Ag in the galvanic series,²⁶⁻²⁷ which impedes electron transfer from titanium to the AgNPs (FIG. 1C). As a result, in comparison with SNPSA materials, SNPT materials had a less positive SP value (FIG. 2A-2E).

The hydrophilicity of the SNPSA surface was significantly increased compared to that of SNPT (Table 2, 3), which can be attributed to the electro-wetting effect.³⁶⁻³⁷ The relationship of the wettability and the SP can be described qualitatively by the following equation (Eq. 6):

$$cos θ ( V ) = cos θ ( 0 ) + \frac{1}{2} \times \frac{∈}{δ \times {γ}_{LV}} {V}^{2} \qquad {( Eq . 6 )}^{36}$$

Here, V is the electrode potential in relation to the potential of the uncharged interface, θ(V) is the contact angle of the coating surface under the external electric field (after coating in this preparation), θ(0) is the contact angle without the external electric field (before coating in this preparation), δ is the thickness of the coating, ε is dielectric constant of the coating, and γ_{LV} is the interfacial tension between liquid/vapor phases. Therefore, the higher SP of SNPSA enhanced its surface hydrophilicity.

The cathodic reaction during the COM treatment is different from the cathodic reaction in a moist environment due to the high amount of water, the high ionic strength, and the physiological pH value (7.2-7.4). The predominant cathodic reactions during the COM treatment are described in the equations below (Eq. 7, 8):

2H₂O+2e⁻→H₂+20H⁻ (Eq. 7)

O₂+4H⁺+4e⁻2H₂O (Eq. 8)

The generated OH⁻ promoted the degradation of PLGA, which led to a rough surface observed by SEM and AFM (FIG. 3A-3C), and more AgNP exposure on the coating surface. At the same time, the PLGA degradation released more H. Therefore, the cathodic reactions that are shown in Eq. 4 and 5 cannot be completely excluded during the COM treatment due to the higher diffusion rate of H⁺ than that of OH⁻ (9.311×10⁻⁵ cm²s⁻¹ vs 5.273×10⁻⁵ cm²s⁻¹).³⁸ Moreover, the SEADI values of SNPSA were much higher than those of SNPT, which indicate that the intensity of the electrochemical reaction on the surface of the SNPSA was much higher than the reaction on the SNPT. Collectively, these post-COM data further confirm our hypothesis that the nanoscale galvanic redox system formed on SNPSA, but not on SNPT.

Traditionally, galvanic reactions between dissimilar metals in direct contact have negative impacts on the surrounding tissue because the continuous electron flow generates elevated oxidation and corrosion of the implants.³⁹ This usually leads to poor implant performance and rejection.³⁹ However, by engineering a controlled galvanic redox reaction on the surface of the 316L-SA, we introduced a novel biological activity—osteogenesis—to a metal material by surface modification alone. Considering that stainless steel alloys comprise the majority of metals used for biomedical bone fixation, are stronger and less expensive than titanium, and account for more than half of the total biomedical metal market,⁴⁰ the newly fabricated SNPSA materials that hold bactericidal and osteogenic dual activities may exhibit particular benefits for orthopaedic and orthodontic applications.²⁸ These enhanced bioactive orthopaedic and orthodontic implants could be particularly applicable in scenarios that require elevated osseointegration and/or built-in electrostimulation. Therefore, this study describes an innovative and highly translational strategy to create osteogenic materials for bone regeneration and opens the possibility of developing materials with significantly improved biological functions.

In agreement with our hypothesis, the AgNP/PLGA coating on different metal substrates, which lead to different electrochemical properties, osteoinductivity, and consequent osseointegration, may also distinguish applications for the metal substrates in vivo. For example, SNPSA materials may be more suitable for permanent intramedullary fixation, especially in scenarios where a large volume of bone tissue is lost and osteoinductivity of the implants is required. Additionally, cases of permanent orthopedic and dental implantation in which osseointegration is crucial, such as joint replacement, prosthetic limbs, and teeth, may find SNPSA particularly useful because of its osteoinductive and antimicrobial properties. On the other hand, although titanium materials usually exhibit good biocompatibility and osseointegration due to the stable oxide layer on its surface,⁴¹ our results demonstrate that introducing a thin AgNP/PLGA coating significantly improves the osseointegration capacity of the less expensive 316L-SA compared to a titanium substrate. The titanium alloy may impart titanium dioxide nanoparticles, which have been reported to alter the viability and behavior of multiple bone related cell types, increase bone resorption, and lead to clinical incidents of osteolysis, implant loosening, and joint pain.⁴² Thus, for fractures without major tissue deficiencies, SNPT materials may be a more desirable choice for external fixation.

### Conclusions

By characterizing the surfaces of the coated metal materials using SEM, EDS, AFM, KPFM, and contact angle measurement, we successfully demonstrated that delicately establishing a nanoscale galvanic redox system to alter the SP of a traditional biomaterial can induce novel bioactivities. For instance, by engineering a nanoscale galvanic redox system between AgNPs and 316L-SA, the AgNP/PLGA coating endowed bactericidal activities to the 316-SA and also introduced novel osteogenic stimulation properties into the system. This markedly advances the orthopaedic and orthodontic applications of SNPSA materials. Importantly, the novel osteoinductivity was only present in the composite materials that could interact in a galvanic redox system, but was not found in the individual components of the composite materials. From the example presented in this study, the AgNP/PLGA coating converted a normally deleterious galvanic redox reaction (e.g., rusting,¹⁷⁻¹⁸ poor implant performance, and rejection³⁹) on metal surfaces into a biological benefit that promoted pen-implant bone growth (data not shown). The universal galvanic redox reaction can also be applied to other metallic materials, such as copper or zinc, and used in orthopaedic, dental, and cardiovascular devices. From these findings, this study enables insight into both the generated electrical forces and potential applications of galvanic redox reactions in biomaterial engineering. We foresee that this study will offer a strong foundation for developing a new class of galvanic redox biomaterials that endow novel biological functions for use in regenerative medicine.

### REFERENCES

(1) Hadi, S.; Ashfaq, N.; Bey, A.; Khan, S. Biological Factors Responsible for Failure of Osseointegration in Oral Implants. Biol. Med. 2011, 3 (2), 164-170.
(2) Subbiandoss, G.; Kuijer, R.; Grijpma, D. W.; van der Mei, H. C.; Busscher, H. J. Microbial Biofilm Growth Vs. Tissue Integration: “The Race for the Surface” Experimentally Studied. Acta Biomater. 2009, 5 (5), 1399-1404.
(3) Raphel, J.; Holodniy, M.; Goodman, S. B.; Heilshorn, S. C. Multifunctional Coatings to Simultaneously Promote Osseointegration and Prevent Infection of Orthopaedic Implants. Biomaterials 2016, 84, 301-314.
(4) Wood, M. R.; Vermilyea, S. G. A Review of Selected Dental Literature on Evidence-Based Treatment Planning for Dental Implants: Report of the Committee on Research in Fixed Prosthodontics of the Academy of Fixed Prosthodontics. J. Prosthet. Dent. 2004, 92 (5), 447-462.
(5) Dalby, M. J.; Gadegaard, N.; Tare, R.; Andar, A.; Riehle, M. O.; Herzyk, P.; Wilkinson, C. D.; Oreffo, R. O. The Control of Human Mesenchymal Cell Differentiation Using Nanoscale Symmetry and Disorder. Nat. Mater. 2007, 6 (12), 997-1003.
(6) Jeon, H.; Schmidt, R.; Barton, J. E.; Hwang, D. J.; Gamble, L. J.; Castner, D. G.; Grigoropoulos, C. P.; Healy, K. E. Chemical Patterning of Ultrathin Polymer Films by Direct-Write Multiphoton Lithography. J. Am. Chem. Soc. 2011, 133 (16), 6138-6141.
(7) Zhang, W.; Liu, N.; Shi, H.; Liu, J.; Shi, L.; Zhang, B.; Wang, H.; Ji, J.; Chu, P. K. Upregulation of Bmscs Osteogenesis by Positively-Charged Tertiary Amines on Polymeric Implants Via Charge/Inos Signaling Pathway. Sci. Rep. 2015, 5, 9369-9379.
(8) Ning, C.; Yu, P.; Zhu, Y.; Yao, M.; Zhu, X.; Wang, X.; Lin, Z.; Li, W.; Wang, S.; Tan, G. Built-in Microscale Electrostatic Fields Induced by Anatase-Rutile-Phase Transition in Selective Areas Promote Osteogenesis. NPG Asia Mater. 2016, 8 (3), e243-250.
(9) Kuzyk, P. R.; Schemitsch, E. H. The Science of Electrical Stimulation Therapy for Fracture Healing. Indian J. Orthop. 2009, 43 (2), 127-131.
(10) Nair, A. K.; Gautieri, A.; Chang, S.-W.; Buehler, M. J. Molecular Mechanics of Mineralized Collagen Fibrils in Bone. Nat. Commun. 2013, 4, 1724-1732.
(11) Black, J.; Baranowski, T.; Brighton, C. Electrochemical Aspects of Dc Stimulation of Osteogenesis. Bioelectrochem. Bioenerg. 1984, 12 (3), 323-327.
(12) Bodamyali, T.; Kanczler, J.; Simon, B.; Blake, D.; Stevens, C. Effect of Faradic Products on Direct Current-Stimulated Calvarial Organ Culture Calcium Levels. Biochem. Biophys. Res. Commun. 1999, 264 (3), 657-661.
(13) Jahn, T. L. A Possible Mechanism for the Effect of Electrical Potentials on Apatite Formation in Bone. Clin. Orthop. Relat. Res. 1968, 56, 261-273.
(14) Wang, Z.; Clark, C. C.; Brighton, C. T. Up-Regulation of Bone Morphogenetic Proteins in Cultured Murine Bone Cells with Use of Specific Electric Fields. J. Bone Joint Surg. Am. 2006, 88 (5), 1053-1065.
(15) Victoria, G.; Petrisor, B.; Drew, B.; Dick, D. Bone Stimulation for Fracture Healing: What's All the Fuss? Indian J. Orthop. 2009, 43 (2), 117-120.
(16) Staicopolus, D. The Role of Cementite in the Acidic Corrosion of Steel. J. Electrochem. Soc. 1963, 110 (11), 1121-1124.
(17) Nishimura, T.; Katayama, H.; Noda, K.; Kodama, T. Electrochemical Behavior of Rust Formed on Carbon Steel in a Wet/Dry Environment Containing Chloride Ions. Corrosion 2000, 56 (9), 935-941.
(18) Ghareba, S.; Omanovic, S. Interaction of 12-Aminododecanoic Acid with a Carbon Steel Surface: Towards the Development of ‘Green’ corrosion Inhibitors. Corros. Sci. 2010, 52 (6), 2104-2113.
(19) Lok, C. N.; Ho, C. M.; Chen, R.; He, Q. Y.; Yu, W. Y.; Sun, H.; Tam, P. K.; Chiu, J. F.; Che, C. M. Silver Nanoparticles: Partial Oxidation and Antibacterial Activities. J. Biol. Inorg. Chem. 2007, 12 (4), 527-534.
(20) Yang, Y. F.; Xue, Y. Q.; Cui, Z. X.; Zhao, M. Z. Effect of Particle Size on Electrode Potential and Thermodynamics of Nanoparticles Electrode in Theory and Experiment. Electrochim. Acta 2014, 136, 565-571.
(21) Zheng, Z.; Yin, W.; Zara, J. N.; Li, W.; Kwak, J.; Mamidi, R.; Lee, M.; Siu, R. K.; Ngo, R.; Wang, J.; Carpenter, D.; Zhang, X.; Wu, B.; Ting, K.; Soo, C. The Use of BMP-2 Coupled—Nanosilver-PLGA Composite Grafts to Induce Bone Repair in Grossly Infected Segmental Defects. Biomaterials 2010, 31 (35), 9293-9300.
(22) Alt, V.; Bechert, T.; Steinrucke, P.; Wagener, M.; Seidel, P.; Dingeldein, E.; Domann, E.; Schnettler, R. An in Vitro Assessment of the Antibacterial Properties and Cytotoxicity of Nanoparticulate Silver Bone Cement. Biomaterials 2004, 25 (18), 4383-4391.
(23) Zhang, S.; Guo, Y.; Dong, Y.; Wu, Y.; Cheng, L.; Wang, Y.; Xing, M.; Yuan, Q. A Novel Nanosilver/Nanosilica Hydrogel for Bone Regeneration in Infected Bone Defects. ACS Appl. Mater. Interfaces 2016, 8 (21), 13242-13250.
(24) Jeong, S. H.; Hwang, Y. H.; Yi, S. C. Antibacterial Properties of Padded Pp/Pe Nonwovens Incorporating Nano-Sized Silver Colloids. J. Mater. Sci. 2005, 40 (20), 5413-5418.
(25) Abidian, M. R.; Kim, D. H.; Martin, D. C. Conducting-Polymer Nanotubes for Controlled Drug Release. Adv. Mater. 2006, 18 (4), 405-409.
(26) LaQue, F. L. Marine Corrosion: Causes and Prevention, Wiley: Hoboken, N.J., 1975.
(27) Forman, C. M.; Verchot, E. A. Practical Galvanic Series, Defense Technical Information Center: Fort Belvoir, 1967.
(28) Liu, Y.; Zheng, Z.; Zara, J. N.; Hsu, C.; Soofer, D. E.; Lee, K. S.; Siu, R. K.; Miller, L. S.; Zhang, X.; Carpenter, D.; Wang, C.; Ting, K.; Soo, C. The Antimicrobial and Osteoinductive Properties of Silver Nanoparticle/Poly (_{Dl}-Lactic-Co-Glycolic Acid)-Coated Stainless Steel. Biomaterials 2012, 33 (34), 8745-8756.
(29) Zheng, Z.; Bei, F. F.; Tian, H. L.; Chen, G. Q. Effects of Crystallization of Polyhydroxyalkanoate Blend on Surface Physicochemical Properties and Interactions with Rabbit Articular Cartilage Chondrocytes. Biomaterials 2005, 26 (17), 3537-3548.
(30) Mittal, K. L. Contact Angle, Wettability and Adhesion, CRC Press: Boca Raton, 2009.
(31) van Oss, C. J.; Chaudhury, M. K.; Good, R. J. Interfacial Lifshitz-Van Der Waals and Polar Interactions in Macroscopic Systems. Chem. Rev. 1988, 88 (6), 927-941.
(32) Hazan, R.; Brener, R.; Oron, U. Bone Growth to Metal Implants Is Regulated by Their Surface Chemical Properties. Biomaterials 1993, 14 (8), 570-574.
(33) Morandi, M.; Banka, T.; Gaiarsa, G. P.; Guthrie, S. T.; Khalil, J.; Hoegler, J.; Lindeque, B. G. Intramedullary Nailing of Tibial Fractures: Review of Surgical Techniques and Description of a Percutaneous Lateral Suprapatellar Approach. Orthopedics 2010, 33 (3), 172-179.
(34) Zhuang, H.; Wang, W.; Seldes, R. M.; Tahernia, A. D.; Fan, H.; Brighton, C. T. Electrical Stimulation Induces the Level of TGF-β1 mRNA in Osteoblastic Cells by a Mechanism Involving Calcium/Calmodulin Pathway. Biochem. Biophys. Res. Commun. 1997, 237 (2), 225-229.
(35) Bodamyali, T.; Bhatt, B.; Hughes, F.; Winrow, V.; Kanczler, J.; Simon, B.; Abbott, J.; Blake, D.; Stevens, C. Pulsed Electromagnetic Fields Simultaneously Induce Osteogenesis and Upregulate Transcription of Bone Morphogenetic Proteins 2 and 4 in Rat Osteoblasts in Vitro. Biochem. Biophys. Res. Commun. 1998, 250 (2), 458-461.
(36) Welters, W. J. J.; Fokkink, L. G. J. Fast Electrically Switchable Capillary Effects. Langmuir 1998, 14 (7), 1535-1538.
(37) Aronov, D.; Rosenman, G.; Karlov, A.; Shashkin, A. Wettability Patterning of Hydroxyapatite Nanobioceramics Induced by Surface Potential Modification. Appl. Phys. Lett. 2006, 88 (16), 163901-163903.
(38) Guseva, O.; Schmutz, P.; Suter, T.; von Trzebiatowski, O. Modelling of Anodic Dissolution of Pure Aluminium in Sodium Chloride. Electrochim. Acta 2009, 54 (19), 4514-4524.
(39) Gittens, R. A.; Olivares-Navarrete, R.; Tannenbaum, R.; Boyan, B. D.; Schwartz, Z. Electrical Implications of Corrosion for Osseointegration of Titanium Implants. J. Dent. Res. 2011, 90 (12), 1389-1397.
(40) Global Biomedical Metal Market, Third ed.; Acmite Market Intelligence: Ratingen, 2014.
(41) Tschernitschek, H.; Borchers, L.; Geurtsen, W. Nonalloyed Titanium as a Bioinert Metal—a Review. Quintessence Int. 2005, 36 (7), 523-530.
(42) Yao, J. J.; Lewallen, E. A.; Trousdale, W. H.; Xu, W.; Thaler, R.; Salib, C. G.; Reina, N.; Abdel, M. P.; Lewallen, D. G.; van Wijnen, A. J. Local Cellular Responses to Titanium Dioxide from Orthopedic Implants. Biores. Open Access 2017, 6 (1), 94-103.

### Example 2

### Stainless Steel Alloy Coating

20-40 nm-diameter spherical silver nanoparticles (QSI-Nano® Silver, QuantumSphere, Inc., Santa Ana, Calif.) were thoroughly mixed with 17.5% (w/v) PLGA (lactic: glycolic=85:15, inherent viscosity: 0.64 dl/g in chloroform; Durect Co., Pelham, Ala.) solution. The proportion of silver nanoparticles refers to the weight ratio of silver nanoparticles to PLGA. 316L stainless steel alloy Kirschner (K)-wire (length: 1 cm, diameter: 0.6 mm; Synthes. Monument, Colo.) and discs (thickness: 1.59 mm, diameter: 6.35 mm; Applied Porous Technologies, Inc., Tariffville, Conn.) were soaked in the silver nanoparticle/PLGA-chloroform solution for 30 s and air-dried completely. The soak-dry process was repeated three times for each SNPSA implant. After incubating for 12 h at 37° C. to ensure a uniform coating, SNPSAs were stored at −20° C. until use. Morphology of the SNPSA was evaluated by scanning electron microscopy (SEM; NovaNano SEM 230-D9064, FEI Company, Hillsboro, Oreg.) (FIG. 1A-1C and FIG. 2A-2E).

### Surface Free Energy

Surface free energy of SNPSAs was obtained from contact angle measurements. Contact angles of multiple standard liquids on the tested SNPSAs were measured using a contact angle analyzer (FTA125; First Ten Angestroms, Portsmouth, Va.). In order to obtain an accurate description of the wetting behavior of various SNPSAs, the surface free energy of the solid (γ_{S}),was considered to be the sum of separate dispersion (γₛ^{d}) and non-dispersion (γₛ^{nd}) contributions. From this two-component model, the following relationship was derived from the dispersion γ^{d} and non-dispersion (also known as ‘polar’) γ^{nd} interactions between liquids and solids.

γ_{L}×(cos θ+1)=2×(γ_{L}^{d}×γ_{shu d})^{1/2}+(γ_{L}^{nd}×γₛ^{nd})^{1/2} (1)

Eq. (1), known as the geometric mean model, allows the calculation of the solid surface free energy using the contact angle (θ) and the surface tension components of the standard liquids, where γ_{L}, γ_{L}^{d}, and γ_{L}^{nd} represent the surface tension and its dispersion and non-dispersion components of the standard liquids, respectively. The surface tension components of the standard liquids are listed in Table 8.

TABLE 8
Surface tension components
of the standard liquids used.

| | Surface tension | | |
|---|---|---|---|
| Standard | components (mN/m) | | |
| liquids | γ_{L} | γ_{L}^{d} | γ_{L}^{nd} |
| Water | 72.8 | 21.8 | 51.0 |
| Glycerol | 64.0 | 34.0 | 30.0 |
| Formamide | 58.0 | 39.0 | 19.0 |
| Ethylene glycol | 48.0 | 29.0 | 19.0 |

### In Vitro Antimicrobial Activity

The Gram-positive vancomycin-intermediate S. aureus (VISA/MRSA) strain Mu50 (ATCC 700699) was cultured in brain heart infusion broth (BHIB; BD, Sparks, Md.) at 37° C.; while biofilm-forming, Gram-negative opportunistic pathogen P. aeruginosa PAO-1 (ATCC 15692) was cultured in Luria Bertani broth (LB; Fisher Scientific, Hampton, N.H.) at 30° C. 10³, 10⁴, and 10⁵ colony forming units (CFU) of bacteria were suspended in 1 ml culture broth and incubated with the SNPSA K-wires at 225 rpm on a shaker for 1, 2, 6, and 24 hours.

At the end of the incubation, Mu50 and PAO-1 bacteria attached to the surface were collected by 0.9% saline solution and plated onto 10-cm BHIB or LB culture medium plates overnight, respectively.

After 18 h incubation, the number of colonies on each plate was counted and the total viable CFU load was determined.

### Ex Vivo Antimicrobial Activity

Femurs isolated from 12-week old male 129/sv mice were used to assay SNPSA antimicrobial activity ex vivo. Briefly, after locating the femoral intercondylar notch, an intramedullary canal was manually reamed into the distal femur with a 25-gauge needle. A SNPSA K-wire was then placed into the intramedullary canal with 2 μl Mu50 or PAO-1 bacteria suspended in phosphate buffered saline (PBS, pH 7.2; Invitrogen, Carlsbad, Calif.).

Femurs with implants were then placed on 100-μm cell strainers (BD) inside 6-well culture plates containing 2 ml α-minimal essential medium (α-MEM; Invitrogen) supplemented with 1% HT supplement (Invitrogen) and fetal bovine serum (FBS; Invitrogen).

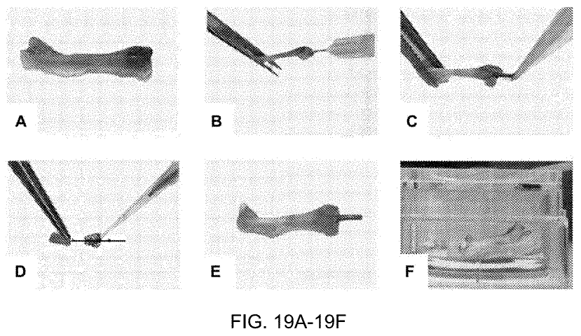

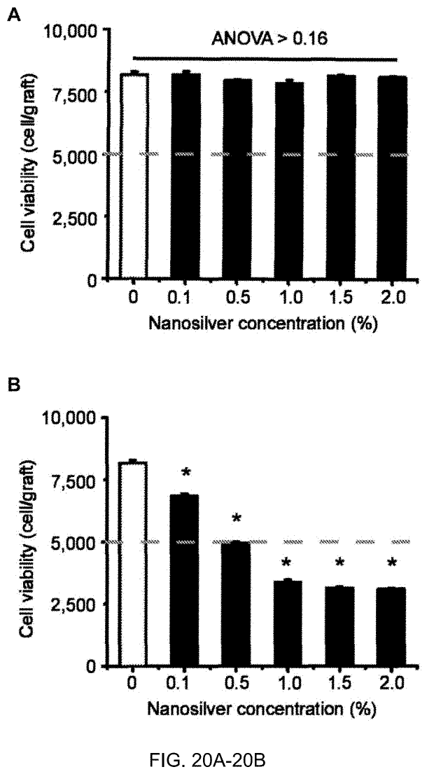

In order to avoid direct contact between SNPSAs and cell culture medium, the distal femur with a protruding SNPSA was angled superiorly, and the proximal femur was soaked in culture medium (FIG. 19A-19F).

After 18 h of incubation at 37° C., 5% CO2, and 95% humidity, SNPSAs were removed from the intramedullary canal and incubated in 1 ml nutrient PBS (1×PBS with 0.25% glucose, 0.2% ammonium sulfate, and 1% sterile bacterial growth broth) for 18 h. 100 μl of released bacteria was transferred into a 96-well microplate and amplified by adding 100 μl fresh bacterial culture broth for another 40 h.

Proliferation of the released daughter cells was monitored at a wavelength of 595 nm using an Infinite f200 microplate reader (Tecan, Durham, N.C.) to generate a time-proliferation curve for each well of the microplate, as previously described.

In this assay, lagging or absent bacterial growth was diagnostic of partial or complete inhibition by the SNPSA, such that only a few or no daughter cells were able to colonize the substrate.

### Protein Adsorption In Vitro

SNPSA discs were incubated at 37° C. for 20 h with 500 μl α-MEM containing 10% FBS and either 0.1 mg/ml bovine serum albumin (BSA; Fisher Scientific) or 0.1 mg/ml BMP-2 (Medtronic, Minneapolis, Minn.). To harvest all adsorbed proteins, SNPSAs were then incubated in 10 mM TRIS (Fisher Scientific) and 1 mM EDTA (Fisher Scientific) for 6 h at 4° C. Protein concentration was measured using the Bio-Rad® Protein Assay (Bio-Rad, Hercules, Calif.) with the Tecan Infinite f200 microplate reader.

### In Vitro Osteoinductivity

2×10³ pre-osteoblastic MC3T3-E1 murine cells (passage 18, subclone 4, ATCC CRL-2593) were seeded on SNPSA discs with 500 μl osteogenic medium (a-MEM supplied with 10% FBS, 1% HT supplement, 100 unit/ml penicillin, 100 μg/ml streptomycin, 50 μg/ml ascorbic acid and 100 mM β-glycerophosphate) in 24-well plates at 37° C., 5% CO₂, and 95% humidity. All media for cell culture were purchased from Invitrogen. Cell proliferation was estimated using the Vybrand® MTT Cell Proliferation Assay Kit (Invitrogen). ALP activity and degree of mineralization (assessed by Alizarin Red staining) were used to quantify the effect of silver nanoparticle/PLGA-coated stainless steel alloy on osteoblastic differentiation.

Rat Fc Model p All surgical procedures were approved by the UCLA Office of Animal Research Oversight (protocol #2008-073). Using aseptic technique, a 25-30 mm longitudinal incision was made over the anterolateral aspect of the left femur of 12-week old male Sprague-Dawley (SD) rats. The femoral shaft was then exposed by separating the vastus lateralis and biceps femoris muscles. Using a micro-driver (Stryker, Kalamazoo, Mich.), four canals were drilled on each femur with 2-mm interface. SNPSA K-wires were implanted into each predrilled canal. For bacterial inoculation, 10³ CFU S. aureus Mu50 or P. aeruginosa PAO-1 in 10 μl PBS (10⁵CFU/ml) was pipetted into the canal before implantation. After inoculation, the overlying muscle and fascia were closed with 4-0 Vicryl absorbable suture to secure the implant in place. Following surgery, the animals were housed in separate cages and allowed to eat and drink ad libitum. Weight bearing was started immediately postoperatively, and the animals were monitored daily. Buprenorphine was administered for 2 days as an analgesic, but no antibiotic was administered.

At 2, 4, 6, and 8 weeks post-surgery, high-resolution lateral radiographs were obtained while the animals were under isoflurane anesthesia. The animals were euthanized at 8 weeks post-implantation. Operated femurs were dissected, harvested, and fixed in 10% buffered formalin (Fisher Scientific). Following 48 h fixation, samples were scanned using high-resolution micro-computed tomography (microCT; Skyscan 1172, Skyscan, Belgium) at an image resolution of 20.0 μm (55 kVp and 181 μA radiation source with 0.5 mm aluminum filter). 2D and 3D high-resolution reconstruction images were acquired using the software provided by the manufacturer.

### Histological and Immunohistochemical (IHC) Evaluation

After 3D microCT scanning, the specimen was decalcified using 10% EDTA solution (pH 7.4, Fisher Scientific, Hampton, N.H.) for 21 days, washed with running tap water for 3-4 h, transferred to a 75% ethanol solution, and embedded in paraffin. 5-μm sagittal sections of each specimen were collected. Hematoxylin and eosin (H&E) staining and Masson's Trichrome staining were used to assess morphology. Taylor-modified Brown and Brenn Gram staining and Giemsa staining were used to assess bacterial contamination and inflammation, respectively. In addition, IHC staining for osteocalcin (OCN, Santa Cruz Biotechnology, Santa Cruz, Calif.) was applied to evaluate new bone generation.

### Statistical Analysis

All results are presented as mean ±standard error of mean (s.e.m.). Statistical significance was computed using one-way ANOVA and independent-samples t-test (Origin 8; OriginLab Corp., Northampton, Mass.). P<0.05 was considered statistically significant. All statistical analyses in this manuscript were conducted per consultation with the UCLA Statistical Biomathematical Consulting Clinic (SBCC).

### Characterization of SNPSAs

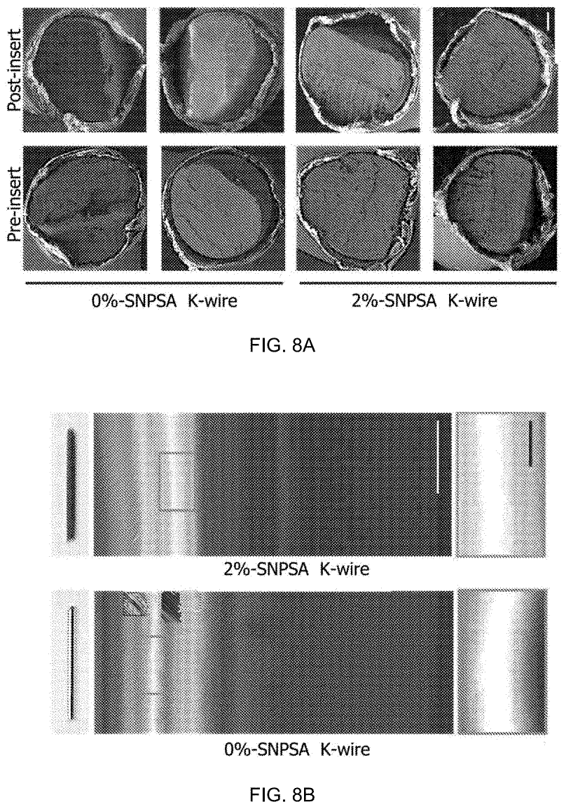

SNPSA was produced by repeated incubations of 316L steel alloy in silver nanoparticle/PLGA-chloroform solution. A uniform layer of silver nanoparticle/PLGA composite was observed on the surface of the stainless steel alloy (FIG. 8A and FIG. 9A-9B and FIG. 9C). In addition, aggregates of silver nanoparticles sintered together were not observed in silver nanoparticle/PLGA layers containing up to 2.0% silver nanoparticles (FIG. 8A, FIG. 9A-9B and FIG. 9C).

SEM revealed that the thickness of silver nanoparticle/PLGA layer coated on K-wires was 43.36±0.08 μm (FIG. 8B; N=8). Densities of coated silver nanoparticle/PLGA composite were 0.263 g/cm³, 0.278 g/cm³, and 0.293 g/cm³, for 0%, 1%, and 2% silver nanoparticles, respectively; thus, the overall doses of silver nanoparticle-coated on the K-wires were: π×[Thickness_{silvernanopartcle/PLGA}+Radius_{Alloy})²−Radius_{Alloy}²]×Density_{silvernanoparticle/PLGA}×Proportion_{silvernanoparticle}=0 μg/cm, 2.44 μg/cm, and 5.14 μg/cm for 0%, 1%, and 2% SNPSA, respectively.

### Contact Angle and Surface Free Energy of SNPSAs

The contact angles on the SNPSAs obtained before and after incubation in osteogenic medium are summarized in Table 9. Notably, the values of contact angle for the liquids applied on 0%-SNPSA differed only slightly before and after incubation in osteogenic medium. In contrast, the values of contact angle for the liquids applied on 1%- and 2%-SNPSAs dramatically changed after the incubation (Table 9).

TABLE 9
Contact angles of the standard liquids on the SNPSAs.

| Silver | | | | |
|---|---|---|---|---|
| proportion | Contact angle θ (°) before incubation^{*} | | | |
| (%) | Water | Glycerol | Formamide | Ethylene glycol |
| 0% | 48.6 ± 0.1 | 51.9 ± 0.1 | 45.1 ± 0.2 | 43.0 ± 0.2 |
| 1% | 49.7 ± 0.1 | 54.0 ± 0.2 | 48.3 ± 0.2 | 44.1 ± 0.1 |
| 2% | 50.3 ± 0.1 | 57.3 ± 0.2 | 50.1 ± 0.2 | 48.7 ± 0.2 |
| Silver | Contact angle θ (°) after incubation | | | |
| proportion | in osteogenic medium^{*} | | | |
| (%) | Water | Glycerol | Formamide | Ethylene glycol |
| 0% | 47.0 ± 0.2 | 52.5 ± 0.1 | 40.6 ± 0.1 | 43.8 ± 0.2 |
| 1% | 36.1 ± 0.2 | 51.4 ± 0.1 | 37.4 ± 0.2 | 37.4 ± 0.1 |
| 2% | 27.9 ± 0.2 | 50.1 ± 0.1 | 35.4 ± 0.2 | 29.2 ± 0.2 |

^{*}Data were shown as mean ± SEM (N = 6)
^{#} SNPSAs were incubated in osteogenic medium for 9 days.

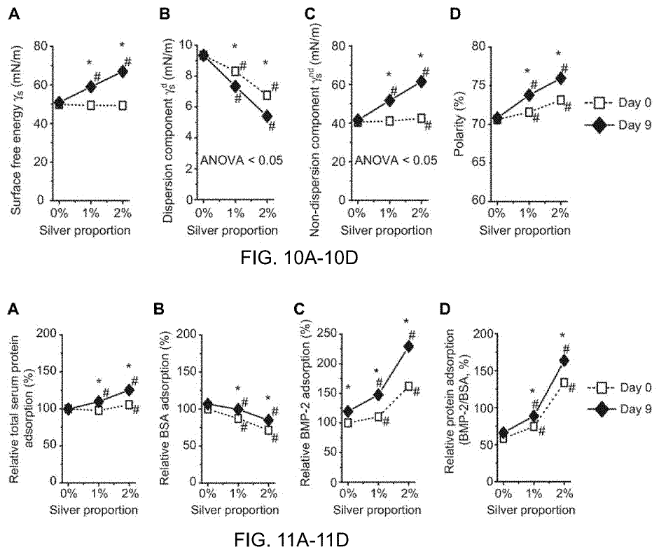

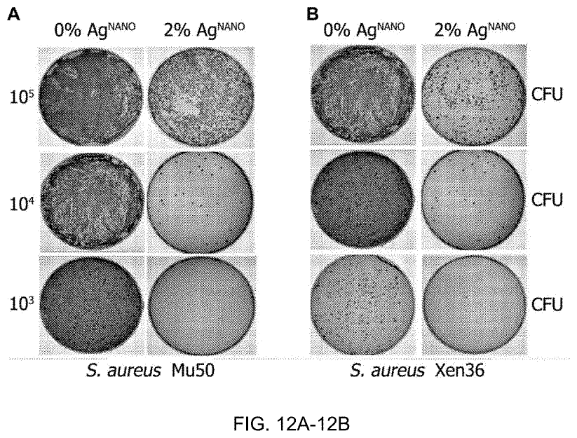

Using the contact angle values and Eq. (1), surface free energy and its dispersion and non-dispersion components of SNPSAs were calculated (FIG. 10A-10D). The presence of silver nanoparticles had minimal effect on the surface free energy of SNPSAs before incubation in osteogenic medium; however, the surface free energy of SNPSAs increased significantly as a function of silver proportion after 9 days of incubation in osteogenic medium (FIG. 10A).

Interestingly, the dispersion component yd decreased with increasing silver proportion (FIG. 10B) but remained quite small compared to the non-dispersion component γₛ^{nd} (FIG. 10C); moreover, incubation in osteogenic medium further decreased γₛ^{d} (FIG. 10B). In contrast, the non-dispersion (or ‘polar’) component γₛ^{nd} increased with silver proportion, and incubation in osteogenic medium resulted in more dramatically increased γₛ^{nd} as a function of silver proportion (FIG. 10C).

As a result, the polarity of SNPSAs, defined as

$$\frac{{Υ}_{s}^{nd}}{{Υ}_{s}} \times 100 \% ,$$

increased with silver proportion (FIG. 10D). It is noteworthy that incubation in osteogenic medium did not influence the polarity of PLGA-coated alloy without silver nanoparticles (0%-SNPSA), but the same incubation resulted in significantly increased polarity of both 1%- and 2%-SNPSAs (FIG. 10D).

### In Vitro Antimicrobial Activity of SNPSAs

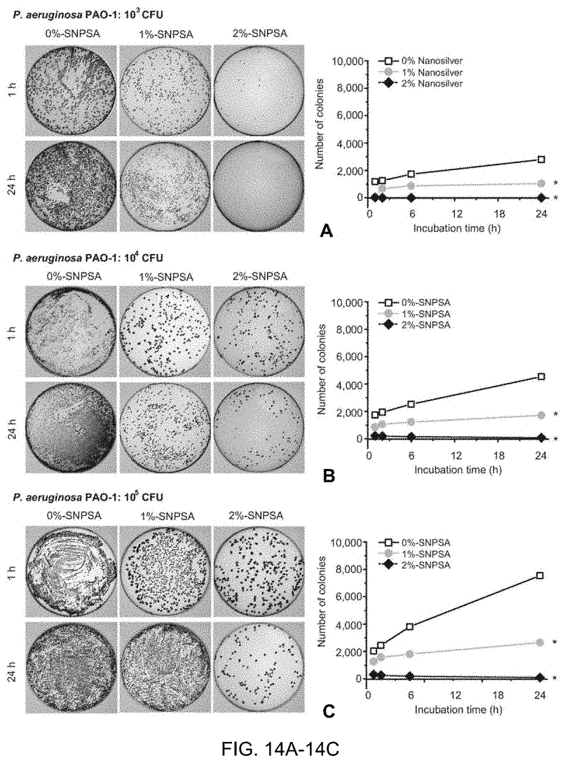

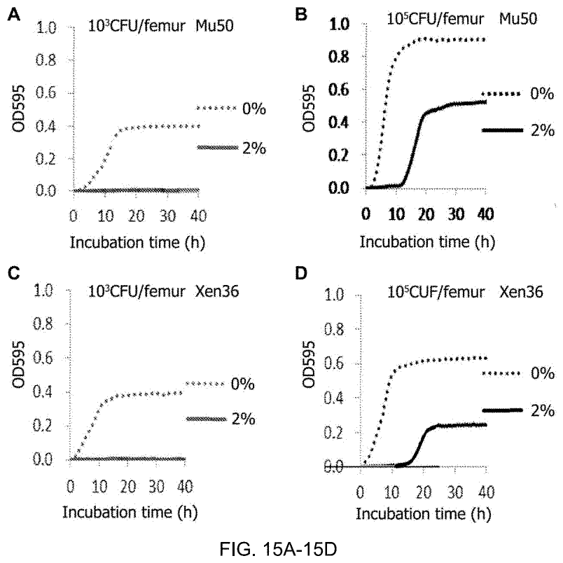

Analysis of bacterial colonization showed that, when compared to 0%-SNPSA, 1%- and 2%-SNPSAs inhibited the initial adherence of S. aureus Mu50 (FIG. 13A-13C) and P. aeruginosa PAO-1 (FIG. 14A-14C) after 1 h incubation in the bacterial broth in a silver-proportion-dependent manner. Quantification of CFU formation demonstrated that, when 0%-SNPSA was incubated with 10³ CFU S. aureus Mu50, almost all the bacteria initially adhered to the alloy surface within the first hour of incubation, and the number of bacteria markedly increased with incubation time (FIG. 13A).

This result suggested that S. aureus Mu50 proliferated extensively on 0%-SNPSA surface after adherence. 1% silver nanoparticles slightly reduced initial adherence of 10³ CFU S. aureus Mu50 but significantly inhibited its proliferation on the coated alloy (FIG. 13A). Initial adherence of 10³ CFU S. aureus Mu50 to 2%-SNPSA was less than 5% (FIG. 13A). Furthermore, no bacteria survived at an initial inoculum of 10³ CFU after 24 h incubation with 2%-SNPSA (FIG. 13A). In addition, 2%-SNPSA presented similar antibacterial properties against the adherent bacteria from 10³ CFU P. aeruginosa PAO-1 as those from the same initial inoculum of S. aureus (FIG. 14A).

When the initial inocula of both species were increased to 10⁴ and 10⁵ CFU, about 2×10³ bacteria initially adhered to the 0%-SNPSA and proliferated during the incubation (FIG. 13B and FIG. 13C, and FIG. 14B and FIG. 14C). In contrast, only about 1×10³ bacteria initially adhered to the 1%-SNPSA, and their extended proliferation was significantly decreased (FIG. 13B and FIG. 13C, and FIG. 14B and FIG. 14C). Remarkably, at the established ceiling of 2% silver, initial bacterial adherence was significantly inhibited (FIG. 13B and FIG. 13C, and FIG. 14B and FIG. 14C). Although 2%-SNPSA was not enough to kill all adherent bacteria from 10⁴ or 10⁵ CFU inoculum within 24 h, less than 1% of adherent bacteria survived (FIG. 13B and FIG. 13C, and FIG. 14B and FIG. 14C).

Ex vivo Antimicrobial Activity of SNPSAs

In order to further evaluate the effect of silver nanoparticle/PLGA coating on preventing bacterial adherence and biofilm formation on the surface of implants, an ex vivo contamination model (FIG. 19A-19F) was employed with a previously reported microplate proliferation assay. The ex vivo model was used to observe the antibacterial activity of SNPSA independently of host immunological responses and to compare its antibacterial activity with that observed in the in vivo contamination model of rat FCs. SEM revealed that placing the SNPSA K-wires into the pre-reamed intramedullary canal did not damage the coating significantly (FIG. 8B).

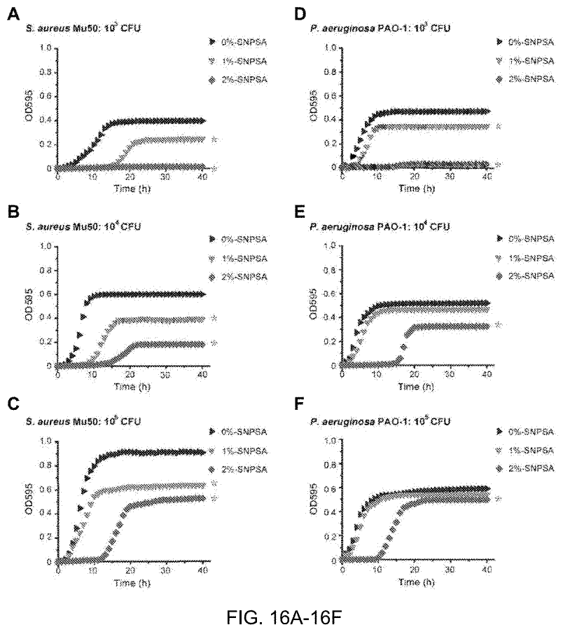

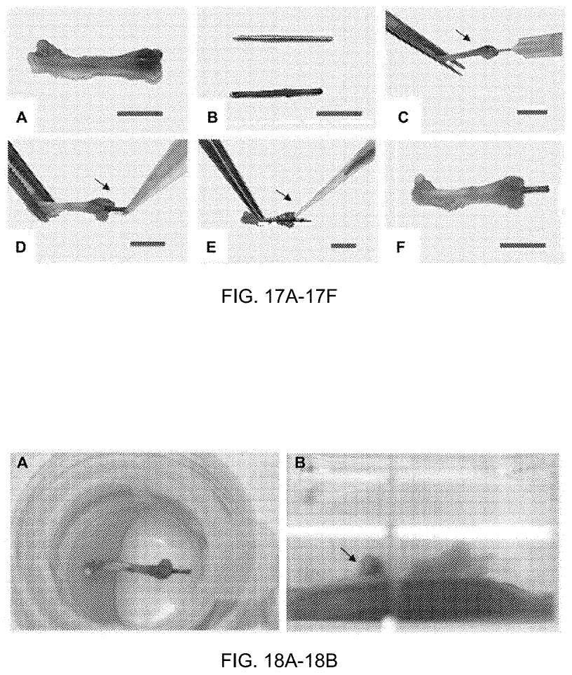

Control 0%-SNPSA did not inhibit ex vivo bacterial adherence or proliferation, while silver-proportion-dependent antimicrobial activity was observed in 1%- and 2%-SNPSAs (FIG. 16A-16F). 1%-SNPSAs significantly inhibited 10³-10⁵ CFU S. aureus Mu50 ex vivo growth on the coated alloy surface (FIG. 16A-16C). However, the inhibition against 10³ CFU P. aeruginosa PAO-1 growth by 1%-SNPSA was minimal (FIG. 16D), and no considerable effects of 1% silver nanoparticle against 10⁴ or 10⁵ CFU P. aeruginosa PAO-1 were observed ex vivo (FIG. 16E, and FIG. 16F). Higher silver proportion at 2% silver nanoparticle was more effective against ex vivo growth of 10⁴ or 10⁵ CFU S. aureus Mu50 (FIG. 16B and FIG. 16C) and P. aeruginosa PAO-1 (FIG. 16E and FIG. 16F), respectively. Furthermore, ex vivo growth of 103 CFU S. aureus Mu50 and P. aeruginosa PAO-1 was completely inhibited by 2%-SNPSA (FIG. 16A and FIG. 16D).

### Protein Adsorption on SNPSAs In Vitro

Protein adsorption was detected on SNPSAs (FIG. 11A-11D). Clearly, a positive correlation between surface free energy and the total serum protein adsorption was observed: the higher the surface free energy, the more protein adsorbed onto the SNPSA surfaces and vice versa (FIG. 10A-10D and FIG. 11A). Surprisingly, SNPSAs exhibited selective protein adsorption in a silver-proportion-dependent manner: as silver proportion increased in SNPSAs, adsorption of the control protein BSA decreased (FIG. 11B) while that of the osteoinductive growth factor BMP-2 increased (FIG. 11C). This selectivity was more significant after the incubation in osteogenic medium (FIG. 11D).

### In Vivo Osteogenic Activity of SNPSAs In Vitro

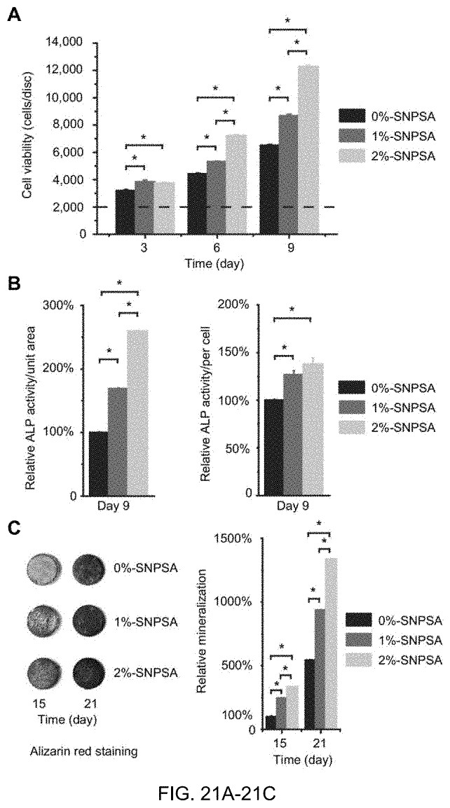

The MTT assay was used to compare mouse MT3T3-E1 pre-osteoblastic cell proliferation on different SNPSAs (FIG. 21A). Generally, silver nanoparticles resulted in increased MC3T3-E1 cell proliferation on SNPSAs in a silver-proportion-dependent manner (FIG. 21A).

Interestingly, along with the culture time, SNPSAs with higher silver proportions promoted cell proliferation more potently (FIG. 21A). For example, cell proliferation on 2%-SNPSA was 1.17, 1.63, and 1.88 times greater than that on control 0%-SNPSA after 3, 6, and 9 days in osteoblastic differentiation medium, respectively. To assay osteoblastic cell function, ALP activity in MC3T3-E1 cells was measured after 9 days in osteoblastic differentiation medium. SNPSAs significantly increased ALP activity of ongrowth cells compared to 0%-silver nanoparticle controls (FIG. 21B).

Furthermore, SNPSAs also significantly promoted ongrowth terminal differentiation of osteoblasts, as indicated by mineralization, during the 21-day culture period (FIG. 21C). Therefore, SNPSAs exhibited osteoinductive properties in a silver-proportion-dependent manner in vitro.

### Effects of SNPSA Implants in Rat FCs

### Radiography

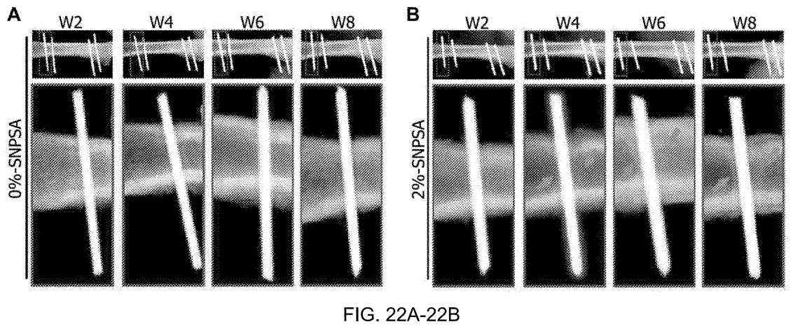

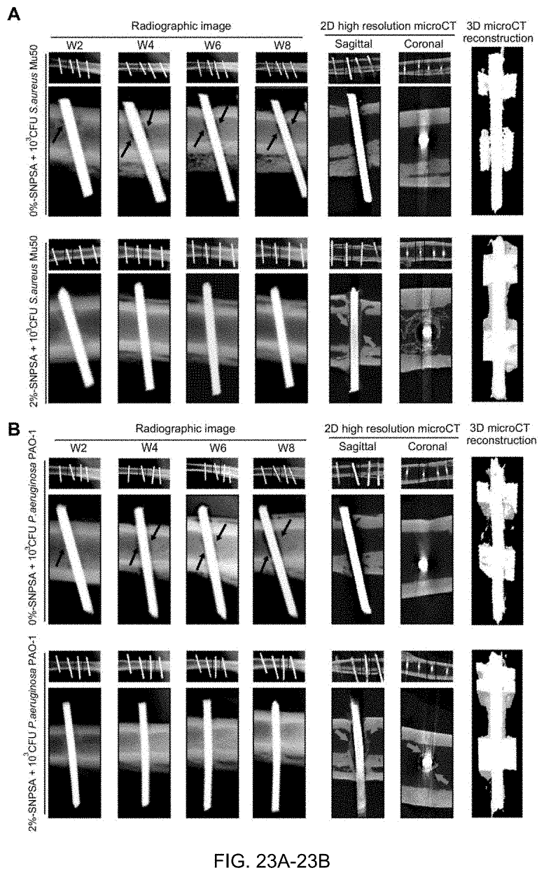

No obvious radiographic signs of bone formation were observed in rat FCs implanted with either uncontaminated (FIG. 22A-22B) or bacterially contaminated (FIG. 23A-23B) 0%-SNPSAs up to 8 weeks post-surgery; instead, radiographic evidence of osseous destruction was detected in the contaminated 0%-SNPSA group (FIG. 23A-23B). In contrast, significant bone formation surrounding 2%-SNPSAs implants in rat FCs was observed despite the initial contamination with 10³ CFU bacteria (FIG. 22A-22B and FIG. 23A-23B). In addition, no osteolysis was observed in the contaminated 2%-SNPSAs group (FIG. 23A-23B). Radiographic findings of bone formation surrounding contaminated 2%-SNPSA implants in rat FCs were also confirmed by 3D microCT analysis (FIG. 23A-23B).

### Histological and IHC Evaluation

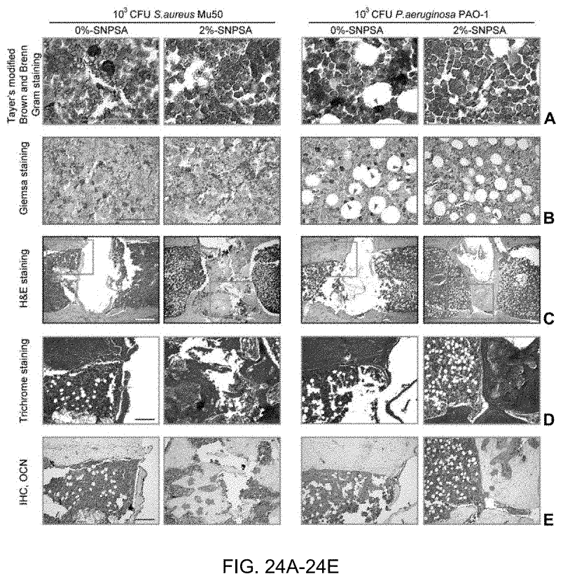

Microscopic examination revealed bacterial persistence (FIG. 24A) accompanied by many inflammatory cells (FIG. 24B) in the intramedullary tissues around 0%-SNPSA implants in rat FCs 8 weeks after implantation with 10³ CFU initial bacterial inoculum. In contrast, no bacterial survival was evident around 2%-SNPSA implants under the same conditions (FIG. 24A), and inflammatory cell infiltration in the intramedullary tissues around the implants was minimal (FIG. 24B). Thus, 2%-SNPSA implants markedly inhibited bacterial invasion without evoking significant host inflammatory responses in vivo.

Newly formed bone around SNPSA implants was further evaluated by H&E staining, Trichrome staining, and IHC staining with an antibody against OCN, a marker of mature differentiated osteoblasts, at 8 weeks after implantation with 10³ CFU initial bacterial inoculum. Only minimal bone formation around the 0%-SNPSA groups was observed (FIG. 24C and FIG. 24D). On the other hand, consistent with radiographic analyses, significant bone formation was detected around 2%-SNPSA implants (FIG. 24C and FIG. 24D), and intense osteocalcin (OCN) staining signified that new bone formation was still active around 2%-SNPSA implants at week 8 after implantation (FIG. 24E). Taken together, 2%-SNPSA implants exhibited significant osteoinductive as well as antibacterial effects in vivo.

Since the first applications of surgically-implanted materials in humans, bacterial infections have represented a common and challenging problem. Bacterial adherence to the foreign implanted materials and subsequent biofilm formation are hallmarks of implant-associated infections. As a result, prevention of bacterial colonization and biofilm formation on implants by administration of prophylactic antibiotics has been extensively studied. Interestingly, most of these studies are focused on preventing S. aureus contamination, as this species is the leading cause of implant-associated infections due to its high affinity to bone, rapid induction of osteonecrosis, and resorption of bone matrix. However, other bacterial species, including P. aeruginosa, S epidermidis, Klebsiella ozaenae, and Escherichia coli, are also commonly involved in implant-associated infections in orthopedic surgery, and some studies have even reported P. aeruginosa as a major isolated organism. Because pathogens involved in implant-associated infections are diverse, and bacteria in biofilms are protected from the host immune responses and antibiotics, the restricted activity of antibiotics against implant infections limits their clinical effectiveness. This is especially the case in infections involving antibiotic-resistant bacterial strains (e.g. MRSA strains), which are increasing in both healthcare and community settings and are becoming a major threat to public health.

Because of its antimicrobial properties, silver has been extensively used in water recycling and sanitization and for treatment of wound infections. Currently, silver is gaining renewed attention as a medical antimicrobial agent due to its broad antibacterial spectrum and the difficulty of developing bacterial resistance to silver. For instance, silver is used to reduce bacterial colonization in a variety of pharmaceutical devices including vascular and urinary catheters, endotracheal tubes, and implantable prostheses. Mechanistically, silver prevents cell division and transcription by binding to and disrupting multiple components of bacterial structure and metabolism, including cellular transport, essential enzyme systems such as the respiratory cytochromes, and synthesis of cell wall components, DNA and RNA; nevertheless, the reservoir form of the active silver form may be diverse. Previously, ionic reservoir forms of silver such as silver nitrate (AgNO₃) and silver sulfate (Ag₂SO₄) have been used to provide protection against bacterial infections. However, despite its effective short-term antibacterial activity, inadequate local retention and severe cytotoxic effects of ionic silver (Ag⁺) have made it undesirable for continually preventing bacterial colonization on the implants. Recent reports have shown that that 20-25 nm silver nanoparticles effectively inhibit microorganisms without causing significant cytotoxicity, and that 10-20 nm silver nanoparticles are nontoxic in mice and guinea pigs when administered by the oral, ocular and dermal routes. These findings suggest silver nanoparticles of the size evaluated in the present study are appropriate for therapeutic application from a safety standpoint.

In addition, the preparation and stabilization of silver nanoparticles remain challenging due to their tendency to aggregate. Several polymers have been used to stabilize silver nanoparticles, including polyethyleneimine, polyallylamine, poly(vinyl-pyrrolidone), and chitosan. The nucleophilic character of these polymers, albeit minor, is sufficient for them to bind to the metal particles by donating electrons.

The US Food and Drug Administration (FDA)-approved, biodegradable and biocompatible polymer PLGA has been chosen in this study because its hydrolysable ester bonds are subject to nucleophilic interactions with incorporated components such as silver particles. Another advantage of PLGA is that it could be applied onto implants using solvent casting techniques, which allow coating of alloys and even plastic surfaces with polished, irregular or porous materials.

For instance, up to 2% silver nanoparticles were coated onto 316L stainless steel alloy within PLGA without aggregation (FIG. 8A, FIG. 8B and FIG. 9C). In addition, PLGA degradation is based on hydrolytic splitting of the polymer backbone into oligomers and release of lactic acid and glycolic acid, two byproducts of various metabolic pathways in the body under normal physiological conditions. Thus, a local delivery system that incorporates silver nanoparticles into the polymer coating ensures not only high local concentrations around the implant for long periods but also reduced risks and side effects for the host organism compared to systemic drug application.

In this study, the results from in vitro and ex vivo assays demonstrated that 2%-silver nanoparticle/PLGA coating effectively prevented bacterial adherence and biofilm formation on the stainless steel alloy implants (FIG. 13A-13C, and FIG. 14A-14C, and FIG. 16A-16F). Using a rat FC model, it was found that 2%-SNPSA displayed significant antibacterial activity against contamination with 10⁵ CFU/ml Gram-positive S. aureus Mu50 or Gram-negative P. aeruginosa PAO-1 (FIG. 23A-23B and FIG. 24A-24E), a bacterial burden typical of invasive tissue infection. In addition, by employing BMP-2-coupled silver nanoparticle/PLGA composite grafts, bone formation was successfully regenerated in a 6-mm critical-sized rat FSD grossly infected with 10⁹ CFU/ml vancomycin intermediate Staphylococcus aureus (VISA)/MRSA strain Mu50. Collectively, the findings support the application of silver nanoparticle/PLGA composite for localized prophylaxis of implant-associated infections.

Notably, surface free energy of SNPSA, especially its non-dispersion component, increases with silver proportion after incubation in osteogenic medium (FIG. 11C). Silver nanoparticles in SNPSA may have contributed to the non-dispersion component of surface free energy by progressively releasing cationic silver [Ag⁺, i.e. ionic silver Ag(I)] and/or exposing partially oxidized silver nanoparticles with Ag⁺chemisorbed to the surface of SNPSA during the incubation.

As a result, the non-dispersion component of surface free energy, the total surface free energy, and the polarity are all increased after incubation in osteogenic medium in a silver-proportion-dependent manner (FIG. 10A-10D). In turn, the increased surface free energy, especially its non-dispersion component, imparts higher bioactivity and increased total protein adsorption to the material after incubation in osteogenic medium (FIG. 11A). Surprisingly, adsorption of BMP-2 on the SNPSA surface is positively correlated with the non-dispersion component of surface free energy, which increases along with the silver proportion and incubation time in osteogenic medium; conversely, adsorption of BSA decreases slightly with increased silver proportion and is not significantly affected by the incubation (FIG. 11B). This result suggests that SNPSAs may have the ability to adsorb proteins selectively in a silver-proportion-dependent manner, which may explain their markedly osteoinductive activity in vitro (FIG. 21A-21C) and in vivo (FIG. 23A-23B and FIG. 24A-24E) when BMP-2 is applied. However, further investigation is necessary to determine the mechanism of this selectivity and the effect of incubation.

In summary, we demonstrated that SNPSA successfully inhibited bacterial adherence and biofilm formation in a silver-proportion-dependent manner. Unexpectedly, we also found that SNPSA materials promoted MC3T3-E1 pre-osteoblast proliferation and maturation in vitro. Finally, we used a rat FC model to show that 2%-SNPSA implants have significantly induced bone generation despite bacterial contamination, even at a bacterial inoculum that could cause invasive tissue infection.

From a materials and device development perspective, SNPSA exhibited strong bactericidal and osteoinductive properties that make it a promising pharmaceutical material in orthopedic surgery. The results also indicated that silver nanoparticle/PLGA coating is a practical process that is non-toxic, easy to operate, and free of silver nanoparticle aggregation. In addition, our results revealed that the antibacterial and osteoinductive activities of SNPSA are silver-proportion-dependent, raising the interest in increasing the silver proportion of the coating in future investigations. Further improvement of interfacial adhesion of silver nanoparticle/PLGA coating to different metal surfaces, such as stainless steel alloys, titanium and titanium-based alloys, and cobalt alloys, should be made for clinical application of silver nanoparticle/PLGA-coated implants in orthopedic surgery, especially when permanent implants such as pins for the fixation of bone fracture are indicated.

Formation of an antimicrobial coating or layer is described in PCT/US2012/061217, the teaching of which, including references cited therein, is incorporated herein in its entirety. For the reason of concise description, the reference listing for Example 2 is omitted, and such references can be found in PCT/US2012/061217.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the specific number of antigens in a screening panel or targeted by a therapeutic product, the type of antigen, the type of cancer, and the particular antigen(s) specified. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

It is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. An implantable device, comprising a galvanic redox system formed on a body substrate of the implantable device, the implantable device having a non-zero surface potential when it is deployed,
wherein the galvanic redox system comprises a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and
wherein:
the first metal site is a layout of the first metal formed on the body substrate or the body substrate itself comprising the first metal;
the second metal site comprises a plurality of particles comprising the second metal; and
the first metal and the second metal form a galvanic redox metal pair (“GRMP”).

2. The implantable device of claim 1, wherein the non-zero surface potential is a positive surface potential.

3. The implantable device of claim 1, wherein the first metal is Fe, Al, Mg, Zn, Cu, Cr, Zr, a stainless-steel alloy, a titanium alloy, a cobalt-chromium alloy, amalgam, or a combination thereof.

4. The implantable device of claim 1, wherein the second metal is Ag, Ti, a silver oxide, a titanium oxide, Au, Pt, or a combination thereof.

5. The implantable device of claim 1, further comprising an antimicrobial component having an optional antimicrobial agent, the antimicrobial component being included in the second metal side of the galvanic redox system or being an additional component deposited on top of the galvanic redox system.

6. The implantable device of claim 1, further comprising a coating formed from a polymer material, wherein the plurality of particles comprising the second metal is inlayed with or embedded within the body substrate of the implantable device or included in the coating.

7. The implantable device of claim 1, wherein the second metal comprises silver (Ag).

8. The implantable device of claim 1, wherein the GRMP is selected from stainless-steel/silver, zinc/silver, zirconium/silver, chromium/titanium, aluminum/titanium, steel alloy/titanium, stainless steel/gold, stainless steel/graphite.

9. The implant device of claim 6, wherein the polymer material comprises poly(lactide-co-glycolide) (PLGA), polylactide (PLA), poly glycolic acid (PGA), polycaprolactone (PCL), poly(3-hydroxybutyrate) (PHB), or a combination thereof.

10. The implant device of claim 1, which is a dental implant, an orthopedic implant, a stent or a cosmetic implant.

11. The implant device of claim 1, wherein the second metal is replaced with graphite.

12. A method of fabricating an implantable device, comprising forming a galvanic redox system formed on a body substrate of the implantable device, the implantable device having a non-zero surface potential when it is deployed,
wherein forming the galvanic redox system comprises forming a first metal site and a second metal site, the first metal site comprising a first metal having a first metal electrode potential (FMEP) and the second metal site comprising a second metal having a second metal electrode potential (SMEP), which FMEP being lower than SMEP and SMEP being substantially different such that the implantable device is galvanized when it is deployed, and
wherein:
the first metal site is a layout of the first metal formed on the body substrate or the body substrate itself comprising the first metal;
the second metal site comprises a plurality of particles comprising the second metal; and
the first metal and the second metal form a galvanic redox metal pair (“GRMP”).

13. The method of claim 12, wherein the non-zero surface potential is a positive surface potential.

14. The method of claim 12, wherein the first metal is Fe, Al, Mg, Zn, Cu, Cr, Zr, a stainless-steel alloy, a titanium alloy, a cobalt-chromium alloy, amalgam, or a combination thereof.

15. The method of claim 12, wherein the second metal is Ag, Ti, a silver oxide, a titanium oxide, Au, Pt, or a combination thereof.

16. The method of claim 12, wherein the implantable device comprises an antimicrobial component having an optional antimicrobial agent, the antimicrobial component being included in the second metal side of the galvanic redox system or being an additional component deposited on top of the galvanic redox system.

17. The method of claim 12, wherein the device further comprising a coating formed from a polymer material, wherein the plurality of particles comprising the second metal is inlayed with or embedded within the body substrate of the implantable device or included in the coating.

18. The method of claim 12, wherein the second metal comprises silver (Ag).

19. The method of claim 12, wherein the GRMP is selected from stainless-steel/silver, zinc/silver, zirconium/silver, chromium/titanium, aluminum/titanium, steel alloy/titanium, stainless steel/gold, stainless steel/graphite.

20. The method of claim 12, wherein the polymer material comprises poly(lactide-co-glycolide) (PLGA), polylactide (PLA), poly glycolic acid (PGA), polycaprolactone (PCL), poly(3-hydroxybutyrate) (PHB), or a combination thereof.

21. The method of claim 12, wherein the implantable device is a dental implant, an orthopedic implant, a stent or a cosmetic implant.

22. The method of claim 12, wherein the second metal is replaced with graphite.

23. A method of treating or ameliorating a medical or cosmetic condition in a subject in need thereof, comprising applying an implantable device according to claim 1 to the subject.

24. The method of claim 23, wherein the subject is a human being.
